(19) United States
(12) Patent Application Publication
(10) Pub. No.: US 2024/0293322 A1
(43) Pub. Date: Sep. 5, 2024

(54) SUSTAINED RELEASE FORMULATIONS COMPRISING A SELECTIVE ANDROGEN RECEPTOR MODULATOR

(71) Applicant: University of Iowa Research Foundation, Iowa City, IA (US)

(72) Inventors: Kathleen A. Sluka, Iowa City, IA (US); Joseph Lesnak, Coralville, IA (US); Aliasger K. Salem, Coralville, IA (US); David S. Nakhla, Iowa City, IA (US)

(21) Appl. No.: 18/572,054

(22) PCT Filed: Jun. 23, 2022
(86) PCT No.: PCT/US2022/034726

§ 371 Date: Dec. 19, 2023

Related U.S. Application Data
(60) Provisional application No. 63/214,095, filed on Jun. 23, 2021.

(51) Int. Cl. A61K 9/16; A61K 31/277; A61K 31/402; A61K 31/404; A61K 31/549; A61P 5/26; A61P 29/00

(52) U.S. Cl. CPC A61K 9/1647; A61K 31/277; A61K 31/402; A61K 31/404; A61K 31/549; A61P 5/26; A61P 29/00

(57) ABSTRACT

A sustained release composition comprising one or more particles comprising a polymer and an amount of a select androgen receptor modulator (SARM), and methods of making and using the composition, are provided.

### CROSS-REFERENCE TO RELATED APPLICATIONS

This application claims the benefit of the filing date of U.S. application No. 63/214,095, filed on Jun. 23, 2021, the disclosure of which is incorporated by reference herein.

### BACKGROUND

A select androgen receptor modulator (SARM) is a drug developed for patients undergoing cancer treatment to minimize bone loss, muscle atrophy, and fatigue (Crawford et. al., 2016; Dalton et al. 2011). The drug works by mimicking the effects of testosterone by binding to androgen receptors to produce effects. Administration of testosterone itself has proven beneficial for preventing general wasting associated with cancer treatment, referred to as anabolic effects. However, delivery of testosterone has many negative side effects such as impaired fertility, virilization, acne and prostate cancer which are known as androgenic effects (Solomon et al. 2019, Mohler et al, 2009). The SARM is unique in that it can selectively bind and activate the androgen receptors associated with anabolic effects while minimizing activation of the androgenic effects of androgen receptor signaling.

Musculoskeletal pain affects 13-47% of the population and costs the United States over $600 billion in health care costs and lost wages (Gaskin & Richard, 2012). Unfortunately, many current analgesics for chronic pain have negative side effects or are addictive; thus, there is an urgent need for the development of safer therapeutics for pain relief. While testosterone, which activates androgen receptors, is analgesic in both animal and human studies (Lesnak et al., 2020; Fanton et al., 2017; Ji et al., 2018; White et al., 2015), there are significant unwanted effects. Medically, those taking testosterone require regular monitoring to monitor adverse events and physiological testosterone levels are maintained. Activation of androgenic pathways by testosterone results in negative side effects such as impaired fertility and virilization (Peterin & Brooks, 2017). Non-steroidal select androgen receptor modulators (SARMs) have tissue specific effects, and thus limit unwanted androgenic effects (Mohler et al., 2009; Solomon et al., 2019; Hanlon et al. 2016). Prior work shows daily SARMs reduce bone loss, muscle atrophy, and fatigue associated with cancer treatment (Dalton et al., 2011; Gao et al., 2004; Gao et al., 2005; Kearby et al., 2007) with minimal side effects and are non-addictive (Mohler et al., 2009; Gao et al., 2004; Crawford et al., 2016). Thus, SARMs have potential to be therapeutic for individuals with chronic musculoskeletal pain.

Daily administration of drugs is associated with reduced adherence and consequent suboptimal dosing. Specifically, adherence to daily administration of prescribed medications is poor with roughly 50% of individuals with chronic diseases including individuals with chronic pain (Jimmy & Jose, 2011; Sampaio et al., 2020; Brown & Bussell, 2011; Timmerman et al., 2016; Kipping et al., 2014). Non-adherence increases with daily dosing frequency of twice (Odds Ratio 1.2-4.8) and three daily administrations (Odds ratio 8.6) when compared with once daily dosing and adherence improves with weekly administration (Odds ratio 1.6-1.9) when compared to daily dosing (Iglay et al., 2015; Johnston et al., 2014). It is estimated that medication noncompliance costs the United States $100 billion per year due to increased healthcare costs, increased morbidity and death (Osterberg & Blaschke, 2005). Further, repeated oral administration leads to fluctuations in drug plasma levels that can lead to subtherapeutic or toxic drug concentrations leading to failure of the treatment or unwanted adverse effects (Sampaio et al., 2020; Timmerman et al., 2016; Kipping et al., 2014; Naguib et al., 2021).

### SUMMARY

The disclosure provides for a Select Androgen Receptor Modulator (SARM)-loaded microparticle (MP) formulation which, in one embodiment, is useful to inhibit or treat pain. The disclosed long-acting SARM-loaded microparticle formulations may avoid the need for the repeated administration of SARM by providing a sustained release of the drug while producing the same effect on pain. Use of a SARM-loaded MP formulation may significantly improve patient convenience, maintain stable blood levels over a prolonged period and improve compliance to treatment. Moreover, the use of a SARM-loaded MP formulation may not result in addictive behavior.

In one embodiment, a sustained release composition is provided comprising one or more particles comprising a polymer and an amount of a SARM, e.g., effective to prevent, inhibit or treat pain or to prevent or treat low testosterone levels, in either males or females. In one embodiment, the particles comprise a synthetic polymer. In one embodiment, the polymer comprises lactic acid, glycolic acid, or a combination thereof. In one embodiment, the particles comprise about 85 to 95% poly lactic-co-glycolic acid (PLGA). In one embodiment, the ratio of lactic acid to glycolic acid is 60:40, 55:45, 50:50, 45:65 or 40:60. In one embodiment, the molecular weight of the PLGA is about 15,000 to about 40,000 MW, e.g., about 24,000 to about 38,000 MW. In one embodiment, the particles are microparticles. In one embodiment, the microparticles have a diameter of about 1 to about 100 microns. In one embodiment, the diameter is about 1 to about 15 microns. In one embodiment, the diameter is about 5 to about 10 microns. In one embodiment, the diameter is about 15 to about 50 microns. In one embodiment, the diameter is about 20 to about 50 microns. In one embodiment, the SARM comprises C-6, S-23, BA321, FL442, MK-45412, LGD226, S-40542, S-1, S-4, GLPG0492, GTx-024 (enobosarm), LY2452473, GSK2881078, GSK2849466, PF-06260414, or LGD-4044. In one embodiment, the composition further comprises a pharmaceutically acceptable carrier. In one embodiment, the SARM in the microparticle is released for up to 10 weeks. In one embodiment, the SARM in the microparticle is released for up to 8 weeks. In one embodiment, the SARM in the microparticle is released for up to 6 weeks. In one embodiment, the SARM in the microparticle is released for up to 4 weeks. In one embodiment, the composition is injectable.

In one embodiment, an injectable composition comprising a long-acting SARM-loaded microparticle, e.g., a PLGA microparticle, formulation is provided which is useful for acute and chronic muscle pain. For example, in one embodiment, one or more SARMs are encapsulated in poly(lactide-co-glycolide) (PLGA) polymer in the form of spherical particles in the micron size (e.g., from about 5 to about 50 μm). PLGA is a biodegradable and biocompatible polymer that degrades into safe, non-toxic, non-inflammatory material when injected in the body. The degradation rate of the polymer is dependent on its chemistry, e.g., the ratio of lactic to glycolic acid monomers, and other factors such as the particle size. As disclosed herein, two formulations were developed with different particle sizes, with formulation 1 having a larger particle size than formulation 2. Formulation 1 released the SARM drug slower than formulation 2 due to the slower degradation of PLGA in formulation 1. These microparticle formulations may be employed for subcutaneous (SC) injection, thereby providing for a sustained release of the loaded SARM microparticle.

The disclosure thus provides for a sustained release formulation comprising a plurality of particles, e.g., microparticles, comprising one or more SARMs. In one embodiment, the sustained release formulation comprises particles formed of a synthetic polymer, such as a poly(lactic-co-glycolic) (PLGA) copolymer, e.g., the polymer in a particle is from about 70% w/w to about 95% w/w, e.g., from about 85% w/w to about 95% w/w, e.g., about 90% w/w. In one embodiment, the synthetic polymer comprises PLGA with a lactic acid to glycolic acid ratio of from about 60:40 to about 40:60, e.g., about 55:45 or 45:55, e.g., 50:50. The one or more compounds including at least one SARM is/are present in a particle (e.g., relative to the total amount of polymer) at about 1% w/w to about 20% w/w, e.g., about 5% w/w to about 15% w/w, including about 10% w/w.

In one embodiment, the sustained release formulation comprises particles formed of a synthetic polymer comprising 80% w/w to less than 100% w/w of a PLGA copolymer with a ratio of lactic and glycolic acids between 0:100 and 100:0, e.g., about 50:50, 45:55, 40:60, 60:40 or 55:45, and from 0.01 w/w to 20% w/w of an active pharmaceutical ingredient, as disclosed herein. In one embodiment, a composition having a plurality of different particles, such as particles having different ratios of lactic acid to glycolic acid, or a plurality of particles having different diameters, for example, microparticles having a diameter of about 5 to about 10 microns and microparticles having a diameter of about 20 to about 50 microns, are envisioned.

The sustained release formulation, in one embodiment, is administered in an amount that prevents, inhibits or treats pain, e.g., chronic pain, or other maladies. In one embodiment, a single dose of a sustained release formulation comprises about 10 to about 500 mg of SARM-loaded microparticles, e.g., for administration to a human. In one embodiment, a single dose of a sustained release formulation comprises about 50 to about 250 mg of SARM-loaded microparticles, e.g., for administration to a human. In one embodiment, a single dose of a sustained release formulation comprises about 75 to about 200 mg of SARM-loaded microparticles, e.g., for administration to a human. In one embodiment, a human is administered, e.g., intramuscularly or subcutaneously, about 25 mg/kg to about 200 mg/kg of a SARM, e.g., about 50 mg/kg to about 175 mg/kg or about 75 mg/kg to about 150 mg/kg.

In one embodiment, a method to prevent, inhibit or treat pain in a mammal, comprising administering to a mammal in need thereof an effective amount of a composition described herein, is provided. In one embodiment, the pain is chronic pain. In one embodiment, the pain is acute pain. In one embodiment, the pain is musculoskeletal pain. In one embodiment, the mammal has fibromyalgia. In one embodiment, the pain is neck, shoulder, or back pain. In one embodiment, the pain is neuropathic pain. In one embodiment, the mammal is a human. In one embodiment, the mammal is a canine, feline, swine, bovine, equine, ovine or caprine. In one embodiment, the composition is injected. In one embodiment, the polymer comprises lactic acid, glycolic acid, or a combination thereof. In one embodiment, the one or more particles comprise about 85 to 95% PLGA. In one embodiment, the ratio of lactic acid to glycolic acid is 55:45, 50:50, or 45:65. In one embodiment, the composition is administered weekly. In one embodiment, more than one dose of the composition is administered. In one embodiment, the composition is subcutaneously administered. In one embodiment, the mammal has osteoarthritis, neuropathic pain or inflammatory pain.

In one embodiment, a method to inhibit or treat chronic pain in a mammal is provided comprising administering to a mammal in need thereof an effective amount of microparticles comprising one or more SARMs. In one embodiment, the mammal is a human. In one embodiment, the composition is injected, e.g., to a neck, back, ankle, hip, knee or shoulder. In one embodiment, the microparticles are formed of lactic acid, glycolic acid, caproic acid, or combinations thereof.

SARMS useful in the compositions and methods include but are not limited to those disclosed in U.S. Pat. No. 8,853,266, WO2007027582, U.S. Ser. No. 10/277,108, U.S. Ser. No. 11/062,752, WO2005000236, WO2008124922, WO2009082437A2, or WO2003077919A1, Chen et al., J. Pharmacol. Exp. Ther., 312:546 (2005), Jones et al., Endocrinol., 150:385 (2009), Watanabe et al., BBRC, 478:279 (2016), Poutiainen et al., Mol. Cell Endrocrin., 387:8 (2014), Schmidt et al., J. Biol. Chem., 285:27054 (2010), Nejishima et al., Prostate, 72:1580 (2012), Gao et al., Endocrinol., 145:5420 (2004), Cozzoli et al., Pharmacol. Res., 72:9 (2013), or Dubois et al., Endocrinol., 156:4522 (2015), the disclosures of which are incorporated by reference herein. In one embodiment, the SARM comprises C-6, S-23, BA321, FL442, MK-45412, LGD226, S-40542, S-1, S-4, GLPG0492, GTx-024 (enobosarm), LY2452473, GSK2881078, GSK2849466, PF-06260414, or LGD-4044.

### BRIEF DESCRIPTION OF THE FIGURES

FIG. 1. SARM reversed muscle hyperalgesia compared with vehicle control.

FIG. 2. Light microscopy image showing the spherical shape of microparticles of formulation 2.

FIG. 3. Light microscopy image showing the microparticle size of formulation 1 of approximately 20 to 50 μm

FIGS. 4A-4B. In vivo release profiles of formulation 1 and 2. Formulation 1 showed a longer lasting increase in SARMs in plasma. Mean+/−SEM (n=8 per formulation)

FIG. 5. In vitro release profiles of formulation 1 and 2. Mean+/−SEM

FIG. 6. SARM microparticle formulation reversed muscle hyperalgesia compared with vehicle control when in an animal model of fibromyalgia. Injections were given 1 week apart and show a reduction in hyperalgesia after the second injection. Data are shown as a change score from pre-injection values. A decrease in withdrawal thresholds occurs within 24 hours after induction of the model (noted as a greater number). *, p<0.05 when compared to MP vehicles. Mean+/−SEM.

FIG. 7. Differential scanning calorimetry thermograms showing a sharp endothermic peak at approximately 130° C. corresponding to the melting point of the SARM which disappeared in both formulations 1 and 2 indicating the encapsulation of the SARM drug in the microparticles.

FIG. 8. Encapsulation drug loading and efficiency for both formulations.

FIGS. 9A-9C. Daily SARM injection reverses muscle hyperalgesia but not cutaneous paw hypersensitivity. Graphical depiction of experimental design. Following induction of the acidic saline muscle pain model, animals received daily subcutaneous injection of SARM (25 mg/kg) or its vehicle for 4 weeks while pain measures were reassessed on days 1, 3, 5, 7, 14, 21, and 28 (A). Daily SARM administration reversed muscle hyperalgesia measured via muscle withdrawal threshold (MWT) on both the ipsilateral and contralateral limb (B). Daily SARM administration was unable to alleviate paw hypersensitivity measured via repeated von Frey application on both the ipsilateral and contralateral hind paw (C). *p<0.05 compared with vehicle; D-day, B-baseline, Veh=vehicle; Data are mean±SEM.

FIGS. 10A-10E. Development of SARM loaded PLGA microparticle formulations. Scanning electron microscope photomicrographs of SARM-loaded PLGA microparticles for formulation 1 and 2 (A). Table depicting drug loading, encapsulation efficiency, percent yield, and average diameter of the two microparticle formulations (B). Particle size distribution histograms of the SARM-loaded PLGA microparticles formulations which show a unimodal gaussian distribution indicating a homogenous particle size distribution (C). Differential scanning calorimetry thermograms of SARM microparticle formulation 2, SARM microparticle formulation 1, blank PLGA microparticles, mixture of SARM and blank PLGA microparticles, and pure SARM (D). Powder X-ray diffraction diffractograms of SARM microparticle formulation 2, SARM microparticle formulation 1, mixture of SARM and blank PLGA microparticles, blank PLGA microparticles, and pure SARM (E). In vitro cumulative release profiles of the SARM-loaded PLGA microparticles for formulations 1 and 2.

FIGS. 11A-11D. SARM-loaded PLGA microparticles alleviates muscle hyperalgesia and works through activation of androgen receptors. Graphical depiction of experimental protocol. Following induction of the acidic saline pain model, animals received subcutaneous injection of SARM loaded PLGA microparticles (60 mg) on day 1, day 7, or on days 1 and 7 or received a vehicle injection. Pain measures were reassessed on days 1, 3, 5, 7, 14, 21, and 28. (A). Animals who received SARM-loaded microparticles (Formulation 1) on days 1 and 7 saw a reversal of muscle hypersensitivity measured via muscle withdrawal threshold (MWT) on both the ipsilateral and contralateral limb when compared to animals receiving vehicle injections. There was no effect on MWT seen for animals who received SARM-loaded PLGA microparticles solely on days 1 or 7 (B). Graphical depiction of experimental protocol. Animals received implantation of a slow release flutamide (200 mg) or vehicle pellets 7 days prior to induction of the acidic saline muscle pain model. Following induction of the pain model, animals received injections of SARM-loaded PLGA microparticles (60 mg) on days 1 and 7 and pain measures were reassessed on days 1, 3, 5, 7, 14, 21, and 28 (C). Flutamide administration prevented the analgesic effects of the SARM-loaded PLGA microparticles on both the ipsilateral and contralateral limb measured via reversal of MWT decrease as compared with animals who received vehicle pellets (D). *p<0.05 compared with vehicle, #p<0.05 compared with flutamide; D=day, B=baseline, Veh=vehicle, MP=microparticles; Data are mean±SEM.

FIGS. 12A-12D. Safety and toxicology screening for animals who received 2 injections of SARM loaded PLGA microparticles. Serum and heart tissue was collected on day 28 from animals who received SARM loaded PLGA microparticles or vehicle injections both on days 1 and 7. Serum levels of biomarkers for liver and cardiac toxicity showed no differences between animals receiving SARM loaded PLGA microparticles or its vehicle. Average serum levels also fell within normal ranges for each biomarker in animals receiving SARM-loaded microparticles as represented by green shaded boxes (A). Histological (H&E staining) examination of the heart tissue following four weeks of treatment did not show differences between animals who received vehicle or SARM loaded PLGA microparticles (B). Body weights measured throughout the 28 days of treatment revealed no differences between animals receiving SARM loaded PLGA microparticles and vehicle injections (C). Condition place preference testing revealed 5 days of SARM pairing (25 mg/kg) produced no preference for the SARM paired chamber (D). Veh=vehicle, MP=microparticle; Data are mean±SEM.

FIGS. 13A-13D. Ultra-violet (UV) absorbance spectrum of SARM showing maximum absorbance at 250 nm (λₘₐₓ=250 nm) (A). Representative HPLC-UV chromatogram of SARM in aqueous samples (50:50 acetonitrile:water, v/v) at concentrations of 1, 5, and 10 μg/mL (B). HPLC-UV calibration curve of SARM in aqueous samples (acetonitrile:water, 50:50, v/v), showing good linearity in the range of 0.1-50 μg/mL (R²=1.0) (C). HPLC-UV calibration curve of SARM extracted from plasma samples showing good linearity in the range of 0.1-50 μg/mL (R²=0.994) (D). Peak are ratio versus SARM concentration curve.

### DETAILED DESCRIPTION

### Definitions

The chemical genera provided herein are intended to be understood as describing “chemically feasible” structures, by which is meant that the structure depicted by any combination or subcombination of optional substituents meant to be recited by the claim is physically capable of existence with at least some stability as can be determined by the laws of structural chemistry and by experimentation. Structures that are not chemically feasible are not within a claimed set of compounds.

The term “alkyl” as used herein refers to substituted or unsubstituted straight chain, branched, or cyclic, saturated hydrocarbon group. The group can have from 1 to 10 carbon atoms, 1 to 6 carbon atoms, 1 to 4 carbon atoms, 2 to 6 carbon atoms, or 2 to 4 carbon atoms. Examples of straight chain alkyl groups include methyl (i.e., CH3), ethyl, n-propyl, n-butyl, n-pentyl, n-hexyl, n-heptyl, n-octyl groups. Examples of branched alkyl include isopropyl, iso-butyl, sec-butyl, t-butyl, neopentyl, and isopentyl. An alkyl can be optionally substituted. The term “heteroalkyl” refers to an “alkyl” in which one or more heteroatom, such as oxygen, nitrogen, or sulfur is included. For example, a heteroalkyl can include a-CH2-O—CH3 (a C2 heteroalkyl), a CH2OH (a C1 heteroalkyl). In various examples, a heteroalkyl corresponds to an alkyl having a single O, N, or S inserted between two carbons or between a carbon and a hydrogen of the alkyl group. The term “alkenyl” refers to a substituted or unsubstituted straight chain, branched, hydrocarbon group that is at least partially saturated and has at least one carbon-carbon double bond. The term “alkynyl” refers to a substituted or unsubstituted straight chain, branched, or cyclic hydrocarbon group having at least one carbon-carbon triple bond.

The term “aryl” as used herein refers to a cyclic aromatic hydrocarbon group. The group can have from 6 to about 10 carbon atoms, 10 to 20 carbon atoms, or about 6 carbon atoms. Examples include phenyl and naphthyl. An aryl can be optionally substituted.

The term “heteroaryl” or “hetaryl” as used herein refers to an aromatic heterocyclic group. The group can have from a ring size of 5 to 10 atoms, 5 to 9 atoms, or 5 to 6 atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl. A heteroaryl can be optionally substituted.

In general, “substituted” and “substituent” refers to an organic group as defined herein in which one or more bonds to a hydrogen atom contained therein are replaced by one or more bonds to a non-hydrogen atom such as, but not limited to, a halogen (i.e., “halo” selected from F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)2, CN, CF3, OCF3, R′, O, S, C(O), S(O), methylenedioxy, ethylenedioxy, N(R′)2SR′, SOR′, SO2R′, SO1N(R′)2, SO3R′, C(O)R′, C(O)C(O)R′, C(O)CH2C(O)R′, C(S)R′, C(O)OR′, OC(O)R′, C(O)N(R′)2, OC(O)N(R′)2, C(S)N(R′)2, (CH2)O-2NHC(O)R′, (CH2)O-2N(R′)N(R′)2, N(R′)N(R′)C(O)R′, N(R′)N(R′)C(O)OR′, N(R′)N(R′)CON(R′)2, N(R′)SO2R′, N(R′)SO2N(R′)2, N(R′)C(O)OR′, N(R′)C(O)R′, N(R′)C(S)R′, N(R′)C(O)N(R′)2, N(R′)C(S)N(R′)2, N(COR′)COR′, N(OR′)R′, C(═NH)N(R′)2, C(O)N(OR′)R′, or C(═NOR′)R′ wherein R′ can be hydrogen or a carbon-based moiety, and wherein the carbon-based moiety can itself be further substituted. For example, R′ group can be hydrogen, C1-C6 alkyl, or phenyl.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH4+ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present disclosure may take the form of salts. The term “salts” embraces addition salts of free acids or free bases which are compounds. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present disclosure, such as for example utility in process of synthesis, purification or formulation of compounds.

Suitable pharmaceutically-acceptable acid addition salts may be prepared from an inorganic acid or from an organic acid. Examples of inorganic acids include hydrochloric, hydroboric, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of presently described compounds, for example in their purification by recrystallization. Salts may be prepared by conventional means from the corresponding compound, for example, by reacting the appropriate acid or base with a compound described herein. The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

The term “non-steroidal” as used herein in the context of chemical structure refers to a compound having a structure other than a sterane (cyclopentanoperhydrophenanthrene), sterol, or androstane structure or derivative thereof. For example, compounds described herein can have non-steroidal core structures. Examples or non-steroidal structures include compounds according to Formula I, Formula II, Formula III, or Formula IV. In various further examples, compounds used herein can have structure other than a structure comprising the carbon skeleton of cholesterol, sterane, sterol, or androstane.

### Exemplary Compositions and Methods

Recently, there has been a push to understand sex differences found in prevalence of chronic pain conditions. Animal research demonstrated that testosterone can protect males and females against development of widespread pain (Lesnak 2020; Fanton et al., 2017; Ji et al., 2018). There has been one study in women with fibromyalgia which found administration of testosterone had positive effects on pain and fatigue (White et al., 2015). However, testosterone administration carries with it many negative side effects which limits its usefulness as a therapeutic agent for this population

SARM is a drug that was developed to reduce bone loss, muscle atrophy, and fatigue associated with radiation cancer treatment. A SARM functions by mimicking the role of testosterone by binding to androgen receptors to produce effects. Administration, e.g., repeated administration of SARM to animals can alleviate musculoskeletal pain more quickly than vehicle drug delivery. SARMs have a half-life of about 4 hours and thus require repeated administration to maintain effective plasma levels. GTx-024 is a SARM that has not been tested in chronic pain populations.

One way to overcome the adherence and fluctuating drug concentrations of orally delivered drugs is with long-acting, injectable microparticle formulations. These preparations allow for controlled drug delivery by slowly releasing drug over time to provide steady plasma levels of drug following a single administration. Poly(lactic acid-co-glycolic acid) (PLGA) nanoparticles are biodegradable polymers used as therapeutic drug delivery devices for long-term release. PLGA particles therefore increase adherence and ensures drug concentrations remain within a therapeutic window to improve treatment success. SARM microparticle formulation would alleviate muscle hyperalgesia in a mouse model of widespread muscle pain.

As disclosed herein, a sustained release (SR) SARM-loaded microparticle formulation is useful when administered, e.g., as a sub-cutaneous (SC) injection, once/twice to mammals, such as humans, with chronic muscle pain and/or fibromyalgia. The use of microparticles to deliver SARMs provides long-term delivery of the drug to maintain plasma levels. This improves adherence to the treatment and provides long-lasting relieve with limited injections.

The composition can be formulated as a pharmaceutical composition and administered to a mammalian host, such as a human patient in a variety of forms adapted to the chosen route of administration. In one embodiment, the composition is locally administered or is administered prophylactically.

In one embodiment, the composition may be administered by injection. Solutions may be prepared in water, optionally mixed with a nontoxic surfactant. Dispersions may also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection may include sterile aqueous solutions or dispersions or sterile powders comprising the active ingredient which are adapted for the extemporaneous preparation of sterile solutions or dispersions. In all cases, the ultimate dosage form should be sterile, fluid and stable under the conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride.

Sterile solutions may be prepared by incorporating the one or more particles in the required amount in the appropriate solvent with various other ingredients, as required. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compound(s) in the composition can be determined by comparing their in vitro activity and in vivo activity in animal models thereof. Methods for the extrapolation of effective dosages in mice, and other animals, to humans are known to the art; for example, see U.S. Pat. No. 4,938,949.

The concentration of the therapeutically or prophylactically effective compound(s) in a composition, may be from about 0.1-25 wt-%, e.g., from about 0.5-10 wt-% or may be about 0.1-5 wt-%, e.g., about 0.5-2.5 wt-%.

The amount of the therapeutically or prophylactically effective compound for use alone in the particle or with other agents may vary with the composition of the polymer, route of administration, the nature of the condition being treated and the age and condition of the patient and will be ultimately at the discretion of the attendant physician or clinician.

### Exemplary Particle Formulations

The disclosed biodegradable particles may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The disclosed biodegradable particles may be prepared by methods known in the art. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, a particle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle for the compounds disclosed herein may be a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle for the compounds disclosed herein may comprise polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed cationic polymer may include, but are not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle for the compounds disclosed herein may comprise a lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

A biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range of materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isopropylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan.

### Pharmaceutical Compositions

The disclosure provides a composition comprising, consisting essentially of, or consisting of the above-described delivery vehicle, e.g., one or more particle(s), and optionally a pharmaceutically acceptable (e.g., physiologically acceptable) carrier. In one embodiment, when the composition consists essentially of the one or more particles and a pharmaceutically acceptable carrier, and optionally additional components can be included that do not materially affect the composition (e.g., adjuvants, buffers, stabilizers, anti-inflammatory agents, solubilizers, preservatives, etc.). In one embodiment, when the composition consists of the one or more particle(s) and optionally a pharmaceutically acceptable carrier, the composition does not comprise any additional components. Any suitable carrier can be used, and such carriers are well known in the art. The choice of carrier will be determined, in part, by the particular site to which the composition may be administered and the particular method used to administer the composition. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. The composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof.

In addition, one of ordinary skill in the art will appreciate that the composition comprising a delivery vehicle, e.g., one or more particle(s), can be present in a composition with other therapeutic or biologically-active agents. For example, factors that control inflammation, such as ibuprofen or steroids, can be part of the composition to reduce swelling and inflammation associated with in vivo administration. Immune system stimulators or adjuvants, e.g., interleukins, lipopolysaccharide, and double-stranded RNA. Antibiotics, i.e., microbicides and fungicides, can be present to treat existing infection and/or reduce the risk of future infection, such as infection associated with gene transfer procedures.

Injectable depot forms comprising biodegradable polymers such as polylactide-polyglycolide may be employed. Depending on the ratio of components in the polymer, and the nature of the particular polymer employed, the rate of drug release can be controlled. Examples of other biodegradable polymers include poly(orthoesters) and poly(anhydrides). Depot injectable formulations are also envisioned which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition may be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, may be useful for administration of the one or more particle(s).

The dose of the therapeutic or prophylactic in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual.

### Routes of Administration, Dosages and Dosage Forms

Both local administration and systemic administration are contemplated. In one embodiment, a composition may be delivered to the back. One or more suitable unit dosage forms comprising one or more particle(s), can be administered by a variety of routes including local. The formulations may, where appropriate, be conveniently presented in discrete unit dosage forms and may be prepared by any of the methods well known to pharmacy. Such methods may include the step of bringing into association the subunit components, e.g., subunits of a polymer or co-polymer, or the polymer or co-polymer, and the drug and optionally liquid carriers, solid matrices, semi-solid carriers, finely divided solid carriers or combinations thereof, and then, if necessary, introducing or shaping the product into the desired delivery system.

The biocompatible delivery vehicle such as one or more particle(s) may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The biocompatible delivery vehicle such as one or more particle(s) may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

In one embodiment, the biocompatible delivery vehicle such as one or more particle(s) may be formulated for administration, e.g., by injection or via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulary agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

Thus, the local delivery of the biocompatible delivery vehicle such as one or more particle(s) can be by a variety of techniques which administer the one or more particle(s) at or near the site of injury or disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

By way of illustration, liposomes and other lipid-containing delivery complexes may be employed as a delivery vehicle.

The pharmaceutical formulations can also take the form of an aqueous or anhydrous solution, e.g., a lyophilized formulation, or dispersion, or alternatively the form of an emulsion or suspension.

In one embodiment, the one or more particle(s) may be formulated for administration, e.g., by injection or infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulary agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

The local delivery of the one or more particle(s) can be at or near the site of injury or disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animal. Non-human animals include all vertebrates, e.g., mammals and non-mammals, such as non-human primates, sheep, dogs, cats, cows, horses, chickens, amphibians, and reptiles, although mammals, such as non-human primates, sheep, dogs, cats, cows and horses are subjects within the scope of this disclosure. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### Exemplary SARMS

Exemplary SARMS for use in the particles include but are not limited to: S-4, GLPG0492, GSK212A, GSK2849466A, GTx-026, JNJ-26146900, JNJ-28330835, JNJ-37654032, LGD2226, LGD-3303, MK-0773, MK-4541, OPK-88004, Ostarine, Enobosarm, GTX024, MK-2866, RAD140, S-1 and S-4, S-101479, S-23, S-40503, S42, SARM-2f, YK11, S-40542, FL442, BA321, GSK2881078, PF-06260414, and LGD-4033.

Other SARMS for use in the particles include but are not limited to: a compound according to Formula I, or a pharmaceutically acceptable salt thereof:

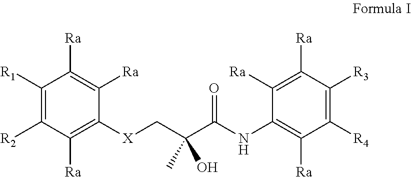

wherein
R1 is F, Cl, Br, I, CF₃, CN, NO₂, NHCORₐ; CONRₐRₐ, SORₐ, or SO₂Rₐ;
R2 is H, F, Cl, Br, I, or CF₃;
R3 is F, Cl, Br, I, NO₂, CN, CF₃, CONRₐRₐ, SORₐ, or SO₂Rₐ;
R4 is H, F, Cl, Br, I, or CF₃;
Ra is, in each instance, independently H, F, Cl, Br, I, CF₃, optionally substituted C₁-C₄ alkyl, or optionally substituted C₁-C₄ heteroalkyl; and
X is O, S, NH, SO2, SO, CH2, or CO.

In one embodiment, the compound Formula I has R1 is CN, NHCOCH3, F, or Cl. In one embodiment, the compound Formula I has R2 is F or H. In one embodiment, the compound Formula I has R3 is CN or NO₂. In one embodiment, the compound Formula I has R4 is CF₃. In one embodiment, the compound Formula I has Ra is H. In one embodiment, the compound Formula I has X is O, SO2, or S. In one embodiment, the compound Formula I has:
R1 is CN, NHCOCH3, F, or Cl;
R2 is H;
R3 is CN or NO₂;
R4 is CF₃;
Ra is H; and
X is O, SO2, or S.

In one embodiment, the compound Formula I has the structure:

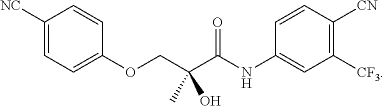 [SMILES: C[C@](O)(COc1ccc(C#N)cc1)C(=O)Nc1ccc(C#N)c(C(F)(F)F)c1]

In one embodiment, the compound Formula I is Enobosarm.

Other SARMS for use in the particles include but are not limited to: a compound according to Formula II, or a pharmaceutically acceptable salt thereof:

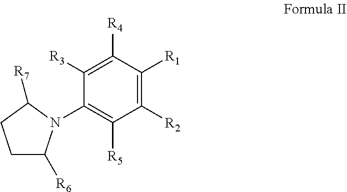

wherein
R1 is F, Cl, Br, I, SRₐ, NO₂, CN, CF₃, CONRₐRₐ, SORₐ, or SO₂Rₐ;
R2 is F, Cl, Br, I, SRₐ, NO₂, CF₃, CORₐ, CO₂Rₐ, CONRₐRₐ, SORₐ, SO₂Rₐ, SO₂NRₐRₐ, NHCORₐ, NHCONRₐRₐ, or optionally substituted C₁-C₄ alkyl,
R3 and R4 are each independently H, F, Cl, Br, I, ORₐ, CF₃, or optionally substituted C₁-C₄ alkyl;
R5 is H, F, Cl, Br, I, CF₃, or optionally substituted C₁-C₄ alkyl;
R6 is optionally substituted C₁-C₈ alkyl, optionally substituted C₂-C₈ alkenyl, CF₃, optionally substituted C₂-C₈ haloalkenyl, optionally substituted C₂-C₈ heteroalkenyl, optionally substituted C₂-C₈ alkynyl, optionally substituted
C₂-C₈ haloalkynyl, optionally substituted C₂-C₈ heteroalkynyl, optionally substituted heteroaryl, optionally substituted aryl, optionally substituted (CH₂)-heteroaryl, optionally substituted (CH₂)-aryl, CH(Rₐ)ORₐ, CH(Rₐ)NRₐRₐ, CORₐ, or Y;
R7 is H, F, CF₃, optionally substituted C₁-C₆ alkyl, optionally substituted C₁-C₆ heteroalkyl, optionally substituted C₂-C₆ alkynyl, and optionally substituted C₂-C₆ alkenyl;
Rₐ is, in each instance, independently H, F, CF₃, optionally substituted C₁-C₄ alkyl, or optionally substituted C₁-C₄ heteroalkyl; and
Y is —CH(OH)CF₃ having an (R)-stereocenter, and which provides an (R)-stereocenter at the carbon to which it is attached.

In one embodiment, the compound Formula II has R1 is CN. In one embodiment, the compound Formula II has R2 is CF₃. In one embodiment, the compound Formula II has R3, R4 and R7 is H. In one embodiment, the compound Formula II has R6 is Y. In one embodiment, the compound Formula II has R6 is Y. In one embodiment, the compound Formula II has the structure:

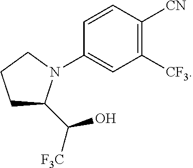 [SMILES: N#Cc1ccc(N2CCC[C@@H]2[C@@H](O)C(F)(F)F)cc1C(F)(F)F]

In one embodiment, the compound Formula II is LGD-4033.

Other SARMS for use in the particles include but are not limited to: a compound according to Formula III, or a pharmaceutically acceptable salt thereof:

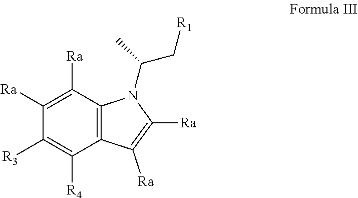

wherein
Ra is, in each instance, independently H, H, F, Cl, Br, I, CF₃, optionally substituted C₁-C₄ alkyl, or optionally substituted C₁-C₄ heteroalkyl;
R1 is NHCOR_{b}, COR_{b}, CONRₐR_{b}, SOR_{b}, or SO₂R_{b}; R3 is CN or NO₂;
R4 is H, F, Cl, Br, I, or CF₃; and
Rb is F, CF3, CN, or optionally substituted C₁-C₄ alkyl.

In one embodiment, the compound Formula III has:
R1 is SO₂R_{b};
R3 CN;
R4 is CF₃;
Ra is H; and
Rb is C₁-C₄ alkyl.

In one embodiment, the compound Formula III has:

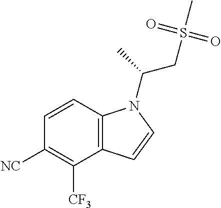 [SMILES: C[C@H](CS(C)(=O)=O)n1ccc2c(C(F)(F)F)c(C#N)ccc21]

In one embodiment, the compound Formula III is GSK2881078.

Other SARMS for use in the particles include but are not limited to: a compound according to Formula IV, or a pharmaceutically acceptable salt thereof:

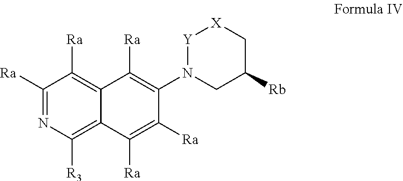

wherein
Ra is H, F, Cl, Br, I, CF₃, optionally substituted C₁-C₄ alkyl, or optionally substituted C₁-C₄ heteroalkyl;
R3 is F, Cl, Br, I, NO₂, CN, CF₃, CONRₐRₐ, SORₐ, or SO₂Rₐ;
Rb is F, Cl, Br, I, CF₃, optionally substituted C₁-C₄ alkyl, or optionally substituted C₁-C₄ heteroalkyl;
X is CH2, absent, or NH; or
Y is NHCOR_{b}, COR_{b}, CONRₐR_{b}, SOR_{b}, or SO₂R_{b}.
36. The composition or method of claim 59, wherein the compound Formula IV has:
Ra is H
R3 is CN
Rb is CH3
X is NH;
Y is SOR_{b}, or SO₂R_{b}.

In one embodiment, the compound Formula IV has the structure:

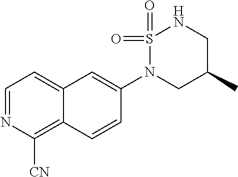 [SMILES: C[C@@H]1CNS(=O)(=O)N(c2ccc3c(C#N)nccc3c2)C1]

In one embodiment, the compound Formula IV is PF-06260414.

The invention will be described by the following non-limiting examples.

### Example 1

A SARM was tested its ability to alleviate pain using a model of fibromyalgia (Sluka et al., 2001). The model is induced by repeated injections of acidic saline into the gastrocnemius muscle, 5 days apart. After the second injection animals develop widespread pain behavior, termed hyperalgesia, that resolves in 4 weeks. Pain-behaviors are tested by using a device to apply deep pressure to the gastrocnemius muscle; withdrawal from this pressure is considered a pain-response.

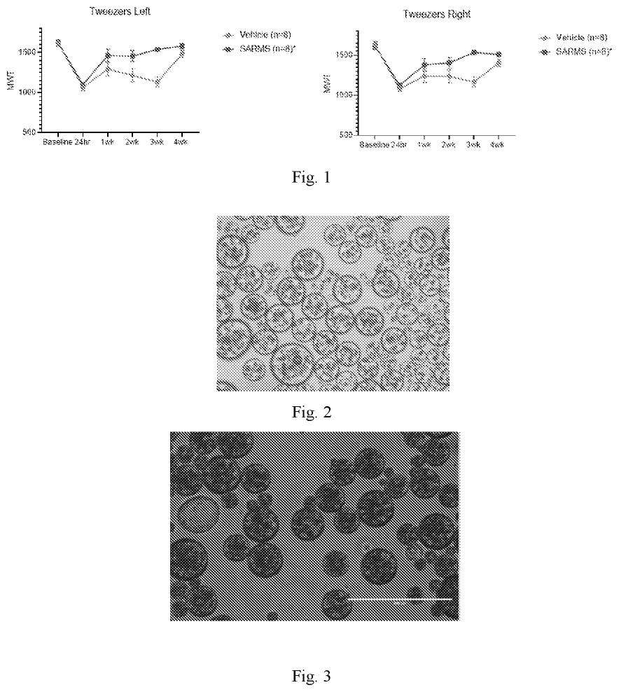

Pain was induced using the model after which daily administration of SARMs or a vehicle was performed for 4 weeks. Withdrawal thresholds of the muscle were tested before and after induction of the model, and weekly after administration of the SARM. A decrease in withdrawal threshold is interpreted as increased pain sensitivity or hyperalgesia. Using a repeated measures ANOVA, the results demonstrated that the animals receiving SARMs (free) had higher pain thresholds than those receiving vehicle treatment for both the left (p=0.006) and right (p=0.009) gastrocnemius muscle (FIG. 1). It is also worth noting that the SARM worked equally in male and female mice. These results demonstrate a use of SARMs in chronic pain patients, particularly fibromyalgia and those with chronic muscle pain. Daily administration is clinically cumbersome and results in lower adherence to the treatment and so a long acting SARM containing microparticle formulation was developed to treat pain, e.g., chronic pain.

Specifically, a long-acting injectable SARM-loaded Poly (D,L-lactide-co-glycolide) (PLGA) microparticle formulation was developed. Sustained release formulations have the advantage of avoiding toxic or subtherapeutic effects that can result from the repeated administration of drugs by providing steady plasma levels over time (Naguib et. al., 2020; Park et. al., 2020; Park et. al., 2019). In addition, sustained release formulations significantly improve patient convenience and adherence to treatment due to the need for a fewer number of administrations. Poly (D,L-lactide-co-glycolide) (PLGA) is a biodegradable, biocompatible polymer that consists of repeating units of lactic and glycolic acid that are bound together through ester linkages. PLGA is widely used as a drug delivery vehicle for encapsulating various small molecules, proteins, and DNA and is generally recognized as safe (GRAS) by the United States Food and Drug Administration (FDA) and the European Medicines Agency (EMA) for administration to humans (Han et al., 2016). An advantage of using PLGA as a drug delivery vehicle is the ability to tailor the release profile of the encapsulated drug, e.g., by controlling the polymer chemistry, specially, the lactide to glycolide ratio (L:G ratio) (Lagreca et. al., 2020). A higher lactide content results in a slower degradation rate of the polymer and an overall slower cumulative release profile.

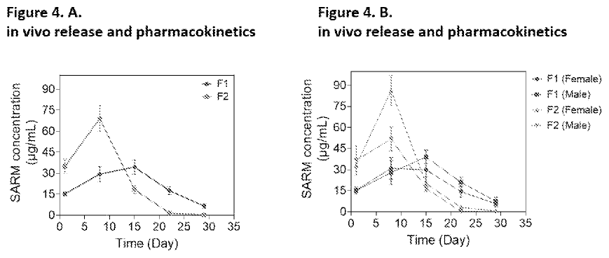

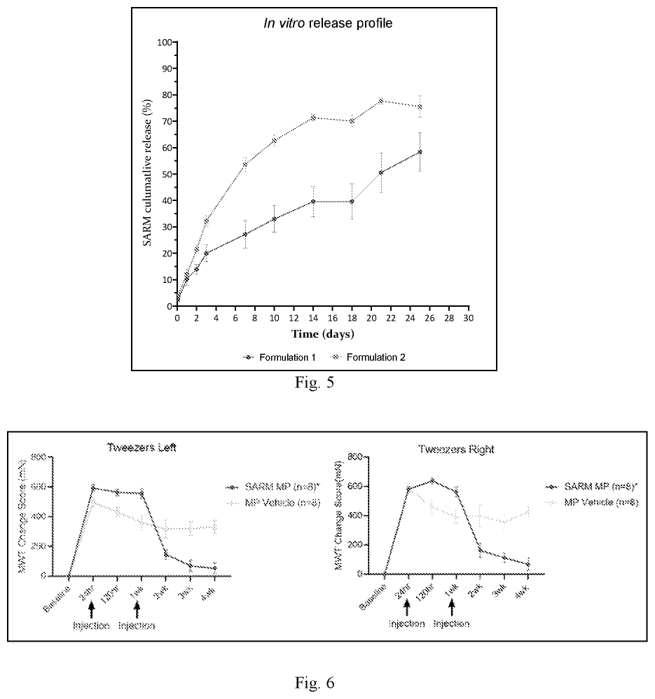

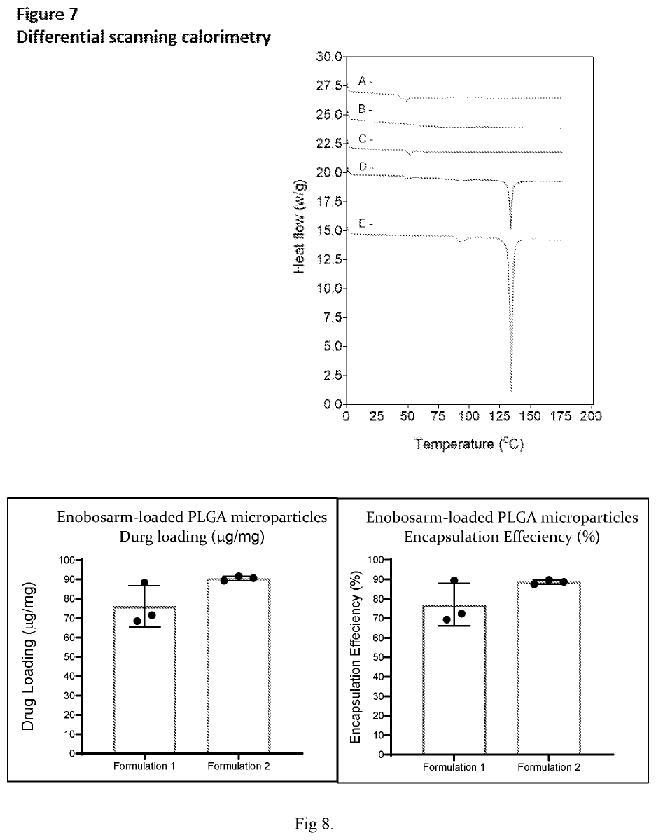

A SARM drug was encapsulated into the PLGA polymer using a single (oil-in-water) emulsion solvent evaporation method. Briefly, SARM and PLGA (Resomer RG 503 H) were dissolved separately in dichloromethane (DCM) at a concentration of 10 mg/mL and 100 mg/mL, respectively. In another vial, 1 mL of each solution was mixed to make a 2 mL organic solution in DCM containing 10 mg SARM and 100 mg PLGA (1:10, drug:polymer ratio). The aqueous phase consisted of 30 mL 1% polyvinyl alcohol (surfactant) in nanopure water. The organic solution was added to the aqueous phase and the mixture was immediately emulsified using either a Talboys Model 101 over head mixer at speed 2833 rpm for 4 minutes (formulation 1) or an overhead homogenizer (Ultra-turrax T25 basic, Ika Works, Inc., Wilmington, NC) at speed 9500 rpm for 1 minute (formulation 2). Formulation 1 and 2 had particle sizes ranging from 20 to 50 μm and 5 to 10 μm, respectively as measured by the light microscope (FIGS. 2 and 3). The in vitro release kinetics of formulation 1 has shown a slower release than that of mice, formulation 1 showed detectable SARM plasma levels up to 1 month, while formulation 2 was only detectable for 3 weeks (FIG. 4). Differential scanning calorimetry thermogram of the SARM drug has shown a sharp endothermic peak at 130° C., which disappeared in formulation 1 and formulation 2 thermograms, indicating the encapsulation of SARM in the PLGA polymer (FIG. 7). Drug loading and encapsulation efficiency of the SARM-loaded microparticles formulation 1 and 2 were calculated using the equations below. Briefly, 2 to 3 mg of the microparticles were dissolved in 1 mL acetonitrile, then 100 ul of that solution was transferred to 900 ul (50:50 0.1% TFA Acetonitrile: 0.1% TFA nanopure water, v/v), then analyzed using high performance liquid chromatography (HPLC-UV) (FIG. 8). In vitro release profiles of both formulation are shown in FIG. 5. Both formulations showed a long-lasting release in vivo but slower and more sustained plasma concentrations were observed with formulation 1 when compared to formulation 2.

Based on in vitro and in vivo data, the microparticle formulations were tested in the fibromyalgia animal model for their ability to reverse hyperalgesia (pain-behaviors). A series of preliminary experiments developed appropriate dosing and delivery schedules for the two formulations. FIG. 5 shows results from delivery of 2 injections, 1 week apart, of SARM microparticle formulation 1. This formulation was injected at 24 hours and 1 week after induction of the model. Withdrawal thresholds of the muscle were tested before and after induction of the model, and weekly after administration of SARM microparticle. In this data the graphs represent differences from baseline so an increase in the Y axis denotes increased pain behaviors (or a lowered threshold to mechanical stimulation). After induction of the model a decrease in withdrawal threshold of approximately 600 mN was observed. After the second injection of SARMs there was a significant increase in the withdrawal threshold, represented as a reduced change score. When compared to SARM microparticles, there was a significant difference between groups, bilaterally (repeated ANOVA; ipsilateral, left (p=0.02); contralateral, right (p=0.01)) (FIG. 6). Again, it was found that the SARM microparticle formulation worked equally as well in male and female mice. These results demonstrate a delivery method for the use of SARMs in chronic pain patients, particularly fibromyalgia and those with chronic muscle pain.

The table below shows the polymer used, emulsifier, amount of drug and size of the particles for the two formulations.

TABLE 1

| | | | | Emulsification | | SARM | Size |
|---|---|---|---|---|---|---|---|
| | Polymer | Polymer | | | Time | amount | estimate |
| | (mg) | type | Emulsifier | Speed | (min) | (mg) | (um) |
| F1 | 100 | PLGA | Paddle | #4 (2833 | 4 | 10 | 20-50 |
| | | RG503H | stirrer | rpm) | | | |
| F2 | 100 | PLGA | Homogenizer | 9500 rpm | 1 | 10 | 5-10 |
| | | RG503H | | | | | |

$$Drug Loading ( \frac{ug}{mg} ) = \frac{Concentration ( \frac{ug}{mL} ) \times volume ( mL )}{Particles weight ( mg )}$$

### Example 2

There is a need for the generation of non-opioid analgesics for chronic pain. The analgesic effects of testosterone have been demonstrated in preclinical and clinical studies. However, treatment with testosterone is not clinically feasible due to adverse effects. Select androgen receptor modulators (SARMs) were developed to overcome adverse effects of testosterone by selectively activating androgen receptors associated with anabolic effects while minimizing activation of androgenic effects. A SARM-loaded microparticle formulation with a long-term release profile was prepared and tested for efficacy in a preclinical model of chronic muscle pain. Chronic muscle pain was induced by 2 intramuscular injections of acidic saline (20 μl, pH 4.0±0.1) spaced 5 days apart were delivered into the left gastrocnemius muscle of C57/BL6J male and female mice. Muscle withdrawal thresholds (MWT) were assessed with force sensitive tweezers applied to the gastrocnemius muscle before and weekly for 4 weeks after induction of the model. Efficacy of SARM ((s)-3-(4-cyanophenoxy)-n-(4-cyano-3-(trifluoromethyl)phenyl)-2-hydroxy-2-methylpropanamide) (Enobosarm (MK-2866) was tested through daily systemic injection of the drug (25 mg/kg, s.c.) or two injections of SARM loaded poly(lactic-co-glycolic acid) (PLGA) microparticles (200 mg, s.c.; 24 hour, 1 week). Drug release profiles from the SARM microparticle formulation was assessed both in vitro and in vivo via HPLC-UV. Liver and cardiac toxicity was analyzed from serum samples from animals receiving SARM microparticles. Statistical analysis for MWT was performed with repeated measures ANOVA compared with vehicle treatment. Both daily administration of SARMs and two injections of SARM microparticles alleviated decreased MWT bilaterally in both sexes (p<0.01). In vitro and in vivo release studies showed SARM was steadily released from microparticles for 4 weeks. Toxicity panels revealed no adverse effects of SARM microparticle treatment. The current study shows SARMs can alleviate muscle pain and SARM loaded microparticles increase clinical utility of administration due to fewer injections and long-term release profiles.

### Example 3

Currently, there is a need for the generation of non-opioid analgesics for treating chronic pain. Preclinical and clinical studies demonstrate the analgesic effects of testosterone. However, treatment with testosterone is not feasible due to adverse effects. Select androgen receptor modulators (SARMs) were developed to overcome these limitations by minimizing activation of androgenic side effects. As disclosed herein, a SARM loaded PLGA microparticle formulation was developed that was able to reverse widespread muscle pain in two injections. In vitro and in vivo release kinetics demonstrated the microparticle formulation had sustained SARM release for 4 weeks. Cardiac and liver toxicology screens demonstrated no adverse effects of SARM microparticles. Finally, antagonism of androgen receptors throughout treatment blocks the analgesic effects of the SARM microparticles. These studies demonstrate SARM microparticles as a potential therapeutic for chronic muscle pain.

### Materials and Methods:

### Study Design

This study was designed as a randomized, blinded, controlled laboratory experiment using male and female C57BL/6J mice. All behavioral experiments were done under blinded conditions and all animals were randomly assigned to groups using a random number generator software program in blocks of 4, stratified by sex. For each experiment, male and female mice were evenly distributed, and multiple replicates were utilized. For each behavioral experiment, preliminary data was utilized to calculate samples sizes with power set at 0.80 and a significance set at 0.05. There were no data that needed to be excluded and no outliers were found. The main goal of this study was to develop a SARM loaded PLGA microparticle formulation that could alleviate muscle pain. The primary endpoint for the following experiments was muscle hyperalgesia measured via muscle withdrawal threshold. Secondary endpoints were paw hypersensitivity and measures of SARM induced toxicity. In initial experiment, the effectiveness of SARMs for alleviation of muscle pain through daily subcutaneous delivery was confirmed. SARMs were shown to alleviate widespread muscle pain in both male and female mice. Next, multiple SARM loaded PLGA microparticle formulations were prepared based on differing size of the particles which would allow us to manipulate release rate of the SARM. A formulation was developed which demonstrated the ability to release SARM both in vitro and in vivo for 4 weeks. Then the analgesic efficacy of this SARM formulation was tested by delivering it subcutaneously in either 1 or 2 doses spaced 1 week apart. 2 injections of the SARM microparticle formulation were able to alleviate widespread muscle pain in male and female mice. Next, it was confirmed that the SARM microparticle formulation was functioning through activation of androgen receptors by providing the androgen receptor antagonist flutamide throughout the treatment window. Lastly, it was demonstrated that two injections of the SARM microparticles were safe and non-toxic. Further methodological details are provided in Materials and Methods section below.

### Mice

All experiments were approved by the University of Iowa Animal Care and Use Committee and were performed in accordance with the National Institute of Health guidelines. A total of 80 C57BL/6J mice (40 male, 40 female) (20-30 g) (8 weeks of age) (Jackson Laboratories, Bar Harbor, ME, USA) were used in the described studies. All mice were housed 4 per cage on a 12-hour light-dark cycle with access to food and water ad libitum unless noted otherwise. All animals were randomly allocated into groups in blocks of 4 stratified by sex with the use of a random number generator. For each experiment, male and female mice were evenly distributed, and multiple replicates were done for each experiment.

### Acidic Saline Muscle Pain

Widespread muscle pain was produced through two intramuscular (i.m.) injections of acidic saline 5 days apart as previously developed by our laboratory (36) (Sluka et al., 2001). On days 1 and 5, mice were anesthetized with 2-4% isoflurane and injected with 20 μl of pH 4.0±0.05 saline into the left gastrocnemius muscle. This model produces bilateral paw allodynia and bilateral muscle hyperalgesia measured at the gastrocnemius muscle which lasts for up to 4 weeks (Sluka et al., 2001; Yokoyama et al., 2007).

### Behavioral Assessments

Paw allodynia was measured through repeated applications of a 2.44 (0.04 g) von Frey monofilament. Animals were placed inside individual small cages on top of a wire mesh and allowed to acclimate to room 60 minutes prior to testing. The von Frey filament was applied to the left and right hind paw 5 times over 10 rounds. The number of withdrawals per round was then averaged for each paw. To prevent behavioral sensitization to testing, 4 minutes was allowed to elapse between each von Frey round of testing. An increase in the number of responses was interpreted as paw allodynia.

Muscle hyperalgesia was assessed as muscle withdrawal thresholds (MWT) by applying force sensitive tweezers to the left and right gastrocnemius muscle as previously described (Skyba et al., 2005; Sluka et al., 2010). Mice were placed headfirst into a gardener's glove with the hind limbs in extension. Measurement of MWT was determined by applying custom built force sensitive tweezers to the gastrocnemius muscle until the animal withdrew its limb or made an auditory response. Both the left and right gastrocnemius muscle were tested and the average of 3 trials was used to determine MWT for each limb. To prevent behavioral sensitization to MWT testing, 5 minutes was allowed to elapse between each assessment. A decrease in MWT was interpreted as muscle hyperalgesia. This pain model produces bilateral muscle hyperalgesia and paw allodynia which lasts for up to 4 weeks (Sluka et al., 2001; Yokoyama et al., 2007). In each of the following experiments von Frey and MWT were assessed on days 1, 3, 5, 7, 14, 21, and 28 after induction of the pain model. On testing days, von Frey assessments were performed first followed by a 30-minute break before MWT determination. On days where both behavioral assessments and SARM administration occurred, von Frey and MWT determination were performed prior to drug delivery. All behavior testing was done in the morning and the tester was blinded to treatment group during pain assessments.

### Select Androgen Receptor Modulator Administration

The SARM, (s)-3-(4-cyanophenoxy)-n-(4-cyano-3-(trifluoromethyl)phenyl)-2-hydroxy-2-methylpropanamide (Achemblock, San Francisco, CA, USA), was used in the following experiments. To test for the analgesic effects of SARM the acidic saline muscle pain model was induced and the presence of hyperalgesia 24 hours after model induction was confirmed. Animals then received daily subcutaneous injections of either SARM or vehicle for the next 4 weeks. For SARM administration, the drug was dissolved in dimethyl sulfoxide (DMSO; Fisher Scientific, Waltham, MA, USA) at a concentration of 50 mg/ml and then diluted in polyethylene glycol 300 (PEG300; Med Lab Supply, Pompano Beach, FL, USA) until a final solution of 10 mg/ml in 80:20 PEG300:DMSO was achieved. Animals were anesthetized with 2-4% isoflurane and SARM (25 mg/kg) or vehicle were delivered subcutaneously. This dose was chosen based on prior literature which found greatest effect at this dose (Gao et al., 2004). During this experiment, SARM solutions were prepared fresh at the beginning of each week.

### Analytical Methods

### High-Performance Liquid Chromatography Method (HPLC-UV)

### SARM UV Spectrum

SARM was dissolved in ethanol at a concentration of 10 μg/mL; 100 μL of that solution was added to a 96-well plate (quartz), in triplicate then the plate was inserted into a UV-microplate reader (Spectramax plus 384, molecular devices, San Jose, CA).

### HPLC-UV

An HPLC-UV method was developed for the quantification of SARM in both aqueous solution and after extraction from mouse plasma, using an Agilent workstation (Agilent Infinity 1100, Santa Clara, CA) coupled with an Agilent diode array detector. A reversed phase Waters Symmetry C18 column (5 μm pore size, 4.6 mm i.d.×150 mm) (Waters, Milford, MA, USA) was used for analysis. Our mobile phase consisted of a mixture of acetonitrile (0.1% trifluoro acetic acid): water (0.1% trifluoro acetic acid)(50:50 v/v) in an isocratic elution mode. SARM was detected at 250 nm wavelength. The flow rate was set at 1 mL/minute and the injection volume was set at 50 μL at room temperature. A stock solution of SARM was prepared in pure ethanol at a concentration of 1 mg/mL and serial dilutions were prepared using acetonitrile:water (50:50 v/v) to construct the calibration curve in the range of 0.1-50 μg/mL. A linear regression equation was fit to the calibration standards.

### Construction of Calibration Curve

To quantify SARM in mice plasma samples, a calibration curve in plasma was calibrated by spiking plasma (90 μl; Na heparin, mouse BALB/C plasma, Innovative research, Novi, Michigan) with 10 μl of SARM stock solutions (dissolved in ethanol in the range of 1-500 μg/mL) to result in a SARM concentration calibration range of 0.1-50 μg/mL. Ivacaftor was selected as internal standard and 10 μL of a 10 μg/mL solution was spiked to the blank plasma samples for a (1 μg/mL) final concentration.

Extraction of SARM from Mouse Plasma

The SARM plasma calibration standards and plasma samples collected from mice from the pharmacokinetics experiment were extracted using an acetonitrile protein precipitation technique. Briefly, 1 mL acetonitrile was added to 100 μL of either the plasma calibration standards or the collected mice plasma samples (spiked with ivacaftor internal standard—10 μL of 10 μg/mL solution), the mixture was vortexed for 5 minutes then incubated on ice for 10 minutes to allow time for plasma proteins to precipitate. Samples were centrifuged at high speed 10,000×g for 5 minutes at 4° C. and supernatant was then transferred to glass tubes and evaporated under a light stream of nitrogen. The residue was reconstituted in 100 μL 50:50 (v/v) acetonitrile:water, vortexed and centrifuged at 14,000×g then 50 μL of the supernatant was analyzed by HPLC-UV.

### Preparation of SARM-Loaded PLGA Microparticles

Due to the hydrophobic nature of SARM a single (oil-in-water) emulsion solvent evaporation technique was selected to prepare the SARM-loaded PLGA microparticles. Formulation parameters are described in Table 2.

TABLE 2

| | | | | Emulsification | | SARM | Size |
|---|---|---|---|---|---|---|---|
| | Polymer | Polymer | | | Time | amount | estimate |
| | (mg) | type | Emulsifier | Speed | (min) | (mg) | (um) |
| F1 | 100 | PLGA | Paddle | #4 (2833 | 4 | 10 | 20-50 |
| | | RG503H | stirrer | rpm) | | | |
| F2 | 100 | PLGA | Homogenizer | 9500 rpm | 1 | 10 | 5-10 |
| | | RG503H | | | | | |

Briefly, SARM and PLGA (Resomer RG 503 H) were dissolved separately in dichloromethane (DCM) at a concentration of 10 mg/mL and 100 mg/mL, respectively. In another vial, 1 mL of each solution was mixed to make a 2 mL organic solution in DCM containing 10 mg SARM and 100 mg PLGA (1:10, drug:polymer ratio). The aqueous phase consisted of 30 mL (1% w/v) polyvinyl alcohol (surfactant) in Nanopure water (Barnstead Thermolyne Nanopure water purification system, Thermo Fisher, Waltham, MA). The organic solution was added dropwise to the aqueous phase and the mixture was immediately emulsified using either a Talboys Model 101 overhead mixer at speed 2833 rpm for 4 minutes (formulation 1 (F1)) or an overhead homogenizer (Ultra-turrax T25 basic, Ika Works, Inc., Wilmington, NC) at speed 9500 rpm for 1 minutes (formulation 2 (F2)). The emulsion was immediately transferred to a magnetic digital stirrer at speed 400 rpm and left for 2 hours at room temperature in a chemical fume hood to evaporate the organic solvent. The particles were collected by centrifugation at 1000×g for 10 minutes (Eppendorf centrifuge 5864 R, Eppendorf North America, Hauppauge, NY) and washed three times by discarding the supernatant, adding 30 mL Ultrapure distilled water (Invitrogen, Waltham, MA), and resuspending the particles at each time. At the last washing step, the particle pellet was frozen in a −80° C. freezer and then dried under vacuum using a lyophilizer (Labconco Free zone 4.5 L-105° C., Labconco, Kansas City, MO).

### Characterization of SARM-Loaded Microparticles: Particle Size and Surface Morphology

The particles size and surface morphology were characterized using a Hitachi S-4800 scanning electron microscope (SEM, Hitachi High Technologies, Ontario, Canada) as described previously (Naguib et al., 2021; Wafa et al., 2019). Briefly, a small amount of the particles was gently spread onto carbon double-adhesive tape mounted on an aluminum stub. To make the particles electrically conductive, an argon beam K550 sputter coater (Emitech Ltd., Kent, U.K) was used to coat the particles with gold (Au) and palladium (Pd), and the SEM photomicrographs of the particles were taken at 5.0 kV accelerating voltage. To measure the average particle diameter, n=100 particles were selected, and the diameter of each particle was manually measured using ImageJ software (NIH, Bethesda, MA); then the mean and standard deviation (SD) of the obtained data were calculated. To estimate the particles size distribution homogeneity, % distribution histograms of the particles' sizes were plotted using GraphPad Prism software (GraphPad, San Diego, CA). To further obtain a metric for the uniformity of particle sizes within each formulation, we performed span analysis, where the diameter below which 90% (D₉₀) of the particles fell was subtracted from the diameter below which 10% of the particles fell (D₁₀) and the result was divided by the diameter below which 50% of the particles fell (D₅₀) (equation 1). The lower the span value the more homogenous the particle size distribution.

$$Span = \frac{( {D}_{90} - {D}_{10} )}{{D}_{50}} \qquad ( 1 )$$

Differential Scanning calorimetry (DSC)

DSC thermograms of 1-SARM, 2-PLGA Resomer RG 503H, 3-SARM microparticle F1, 4-SARM microparticles F2, and 5-mixture of SARM and PLGA, were obtained using a TA DSC instrument (TA Instruments model Q20, New Castle, DE) coupled with a refrigerated cooling system (RCS90). Approximately, 3-5 mg of each sample was accurately weighed and transferred to a standard Tzero aluminum pan, then covered and compressed with a Tzero lid. The reference sample was an empty aluminum pan covered with a Tzero lid. Pure dry nitrogen purge gas was set at 20 psi pressure and 40 mL/min flow rate. The scanning temperature for all samples was set in the range of 0-175° C. at a 10° C./minute heating rate.

### Powder X-Ray Diffraction (PXRD)

To obtain powder X-ray diffraction patterns of 1-SARM, 2-PLGA resomer RG503H, 3-SARM microparticles (F1 and F2) and 4-Mixture of SARM and PLGA, we used a Siemens D5000 diffractometer. The X-ray source was composed of Cu K_{α} X-rays with λ=1.51418 Å. The diffractograms of the 4 samples were recorded in the range 5° to 50° at 2θ values using a step size of 0.02° and a dwell time of 0.5 seconds.

### Drug Loading, Encapsulation Efficiency, and Yield Percentage

To measure the drug loading (DL) and encapsulation efficiency (EE) of the SARM-loaded PLGA microparticles, approximately 2-5 mg of the microparticles was accurately weighed and dissolved in acetonitrile, then 10- and 100-fold dilutions of that solution were made using 50:50 (v/v) acetonitrile:water and then SARM concentration was determined using our developed HPLC-UV method. Drug loading, encapsulation efficiency and yield percentages of the particles were determined using equations 2, 3, and 4, respectively.

$$Drug Loading ( \frac{ug}{mg} ) = \frac{SARM Concentration ( \frac{ug}{mL} ) \times volume ( mL )}{Particles weight ( mg )} \qquad ( 2 )$$

$$Encapsulation efficiency ( \% ) = \qquad ( 3 )$$

$$\frac{Drug loading ( \frac{ug}{mg} ) \times Total weight or YIELD of particles ( mg )}{Initial amount of drug added ( ug )} \times 100$$

$$Yield ( \% ) = \frac{Weight of Lyophilized particles ( mg )}{Weight of starting materials ( mg )} \times 100 \qquad ( 4 )$$

### In Vitro Release Kinetics

To study the in vitro release kinetics of SARM from the 2 microparticles formulations (F1 and F2), 5 mg of the SARM-loaded microparticles were suspended in 5 mL release medium (n=3 per formulation, total=6 tubes). To prepare the release medium, 5 phosphate buffered saline (PBS) tablets were dissolved in 1 L Nanopure water and 0.4 mL polysorbate 80 was added to that solution to make a 0.4% v/v polysorbate 80 in 1 M PBS, pH=7.4 release medium.

Tubes were transferred to an orbital shaker (New Brunswick Scientific, Edison, NJ) and shaken at 300 rpm at 37° C. Then, at 2, 24, 48, and 72 hours, as well as 7, 10, 14, 18, 21, and 25 days, tubes were centrifuged at 4500×g for 5 minutes, 1 ml sample was withdrawn to be analyzed by HPLC; release medium was discarded carefully and replaced with fresh 5 mL release medium.

### In Vivo Release Kinetics

The in vivo release and pharmacokinetics of the SARM-loaded PLGA microparticles (F1 and F2) were studied in a total of n=16 C57BL/6J mice (4 male and 4 female mice per formulation). An amount of the SARM-loaded PLGA microparticles equivalent to 4 mg SARM based on the drug loading of the particles was accurately weighed and suspended in 0.4 mL 1× Dulbecco's phosphate buffered saline (1×DPBS) then injected subcutaneously (SC) to deliver a dose of 200 mg/kg SARM. SARM-loaded PLGA microparticles were administered at Day 0 and Day 7 of the experiment. At 5 time points, (Days 1, 5, 8, 15, 22, and 29), mice were injected with 100-150 μL ketamine/xylazine (87.5/12.5 mg/kg) solution intra-peritoneally, and a maximum of 0.25 mL of blood were collected via sub-mandibular bleeds and transferred to pre-heparinized tubes. Blood samples were centrifuged at 5000×g and the supernatant (plasma) was separated and stored in −80° C. freezer until ready to be processed and analyzed by HPLC-UV.

### SARM-Loaded Microparticle Administration

To assess the analgesic effects of the SARM microparticles, the acidic saline muscle pain model was induced and the presence of hyperalgesia 24 hours after model induction was confirmed. 60 mg of SARM microparticles dissolved in 400 μl of Dulbecco's phosphate-buffered saline (DPBS; Gibco, Waltham, MA, USA) was administered. Animals were anesthetized with 2-4% isoflurane via vaporizer and injected subcutaneously with either SARM-loaded microparticles or vehicle (DPBS) subcutaneously. Animals received SARM-loaded microparticles at 24 hours, at 1 week, or at both 24 hours and 1 week following induction of the pain model.

### Toxicity

To ensure the overall health of the mice subsequent to SARM-loaded microparticle administration, body weights were assessed throughout the 4 weeks of behavior testing. Animals were weighed twice per week for the duration of the experiment. To test for toxicity, cardiac and liver toxicity was measured after treatment with SARM-loaded microparticles in the animals who received two injections of the microparticles. After the completion of behavior testing (end of week 4), animals were euthanized, and blood was collected via a cardiac blood draw. Blood was placed in 3.0 mL serum blood collection tubes (BD vacutainer, Franklin Lakes, NJ, USA) and allowed to clot for 30 minutes. The tubes were centrifuged at 1000×g for 10 minutes at room temperature, serum was collected, and frozen. Frozen serum samples were sent to IDEXX (Columbia, MO, USA) for analysis of cardiac and liver toxicity panels. These panels detected serum levels of creatine kinase, alkaline phosphatase, aspartate transaminase, alanine aminotransferase, total bilirubin, albumin, and total protein. Immediately after collection of blood, the heart was collected and fixed in 10% neutral buffered formalin and routinely dehydrated through a series of ethanol baths, paraffin embedded and sectioned at about 4 μm onto glass slides for hematoxylin and eosin (HE) staining. Stained tissue sections were given to an ACVP boarded veterinary pathologist for examination.

### Blockade of Androgen Receptors

The androgen receptor antagonist flutamide was used to block androgen receptors during SARM-loaded microparticle formulation administration. Slow release flutamide (200 mg/pellet, 60-day release; Innovative Research of America, Sarasota, FL, USA) or control pellets were implanted subcutaneously. For implantation, animals were deeply anesthetized with 2-4% isoflurane. The pellet was implanted through a small incision at the nape of the neck. The incision was stitched closed with synthetic non-absorbable monofilament silk sutures. To protect the surgical site, animals were placed in single housed cages for the remainder of the experiment. Animals were allowed 7 days to recover from surgery prior to induction of the pain model. This dose was chosen based on prior literature showing that it blocked the analgesic effects of activating androgen receptors (Lesnak et al., 2022).

### Rewarding Behavior

To test if SARM produced rewarding behavior condition place preference testing was employed using a 9-day testing paradigm. The boxes used for testing were 27.5″ by 8.25″ divided into two 10.75″ by 8.25″ pairing chambers and a 4.75″ by 8.25″ middle chamber. The pairing chambers had different colored walls (solid black vs black and white stripe) and floor textures (smooth vs ridged). The middle chamber had solid black walls with smooth floors and was illuminated. Acclimation and baseline testing occurred over the first 3 days. Animals were placed in boxes with access to all 3 chambers for 15 minutes. On the third day, time spent in each box was recorded. Drug pairing then occurred for 5 days on days 4 through 8. Each day, animals were injected subcutaneously with SARM vehicle (80:20; PEG300:DMSO) and placed in a pairing chamber for 60 minutes. Animals were then returned to their home cages. Four hours later animals were subcutaneously injected with SARM (25 mg/kg) and were confined to the opposite pairing chamber for 60 minutes. Chamber pairings were counterbalanced amongst animals. Following pairing, on day 9, animals were placed in boxes with access to all 3 chambers for 15 minutes with time spent in each box recorded. In order to determine if rewarding behavior was present, the amount of time spent in SARM paired chambers was compared between baseline and post drug pairing.

### Statistical Analysis

All data is presented as mean±SEM. Statistical analyses were performed on SPSS Version 25.0 (SPPS Inc. Chicago, IL) and GraphPad Prism Version 7.00 (GraphPad Software, La Jolla, CA). For MWT and von Frey data, a repeated measures ANOVA the data points between day 1 and day 28 with baseline as a covariate was used to determine differences between groups. When appropriate, a Tukey's post hoc test was used to determine group differences. For MWT and von Frey data sets, Mauchly's test of sphericity was implemented. If sphericity of data was not assumed, a Huynh-Feldt adjustment was utilized. However, in all data sets where a Huynh-Feldt was utilized the significance of the results did not change. Therefore for clarity, the uncorrected F and p values are reported in the results section. An un-paired, student's t-test was used to compare the average particle diameter between SARM-loaded microparticle formulation 1 and formulation 2. For serum cardiac and liver toxicant levels, an un-paired student's t-test with a Bonferroni correction for multiple comparisons was used to determine differences between groups. For CPP data, a paired student's t-test was used to compare time spent in SARM paired chamber between baseline and post drug pairing.

### Results

### Daily SARM Administration Alleviates Muscle Hyperalgesia

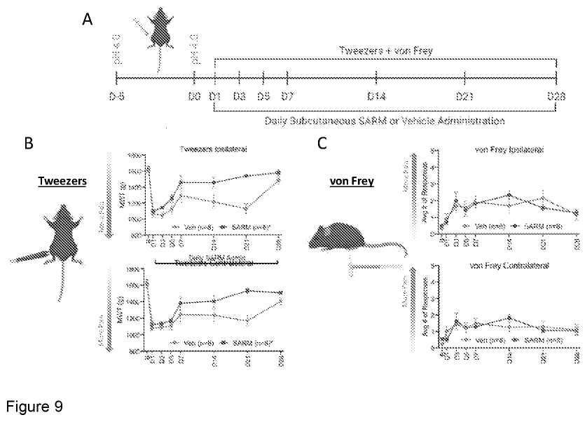

To determine if SARM administration could alleviate pain, the acidic saline muscle pain model was induced, and the presence of muscle hyperalgesia and paw allodynia was confirmed 1 day after model induction. Animals were then randomly allocated to receive daily administration of SARM (25 mg/kg) (n=4 male, n=4 female) or its vehicle (n=4 male, n=4 female) subcutaneously. Muscle hyperalgesia and paw allodynia was reassessed on days 3, 5, 7, 14, 21, and 28 after induction of the pain model (FIG. 9A). Daily SARM administration caused a significant reversal in muscle hyperalgesia measured via MWT on both the ipsilateral (F_{6.78}=3.46, p<0.01) and contralateral (F_{6.78}=3.51, p<0.01) limb when compared to animals receiving vehicle injections (FIG. 9B). However, SARM administration had no effect on paw allodynia as measured via von Frey filament number of responses on both the ipsilateral (F_{6.78}=0.77, p=0.59) and contralateral (F_{6.78}=1.54, p=0.17) paw when compared to animals receiving vehicle injections (FIG. 9C). This demonstrates that SARM has analgesic effects and can alleviate widespread muscle hyperalgesia in an animal model of acid saline induced muscle pain.

### High Performance Liquid Chromatography (HPLC-UV)

### SARM UV Spectrum

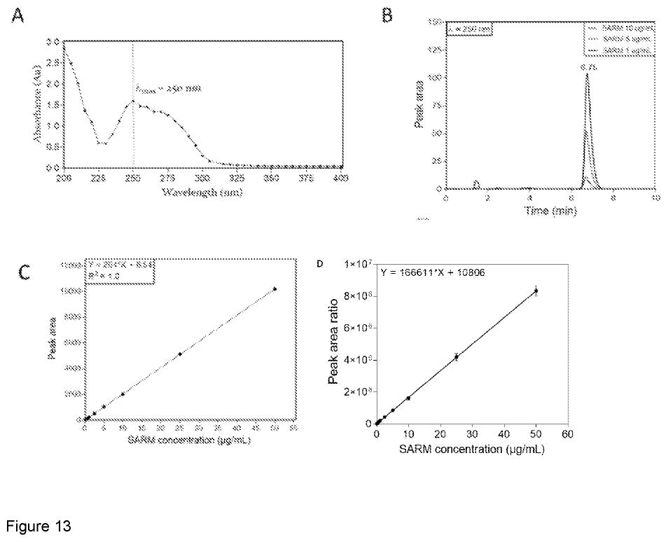

The SARM showed a maximum UV absorbance at 250 nm (FIG. 13A). This is due to the highly aromatic structure of the SARM. This is also in line with SARM UV absorbances reported in literature, however the small variation in the reported wavelengths (270 and 297 nm) could be due to analyzing different batches of SARM using different UV absorbance instruments (Wu et al., 2006; Miklos et al., 2019). This wavelength (250 nm) was selected to quantify SARM using our developed HPLC-UV method.

### HPLC-UV Method and Calibration Curves

SARM showed a retention time of 6.75 minutes (FIG. 13B) and was accurately quantified in aqueous and plasma extracted samples in the linear range of 0.1-50 μg/mL with R² values of 1.0 and 0.99 in aqueous samples and after extraction from spiked blank mice plasma, respectively (FIGS. 13C+13D).

### Preparation of SARM-Loaded PLGA Microparticles

SARM-loaded PLGA microparticle formulations were prepared using a single (oil-in-water) solvent evaporation method. This method was chosen due to the hydrophobic nature of SARM (log P=2.7) (NCBI, 2022). DCM was selected as the inner oil phase to dissolve both the PLGA polymer and SARM. This is because of the universal solvent properties of DCM and its ability to dissolve various PLGA polymers regardless of the lactide:glycolide (L:G) monomer ratio, in addition to its fast evaporation rate due to its low boiling point (39.8° C.) (Park et al., 2019). Polyvinyl alcohol (PVA 1% w/v) was selected as the surfactant to stabilize the oil-in-water emulsion. This is due to its ideal physicochemical properties including its biodegradability, biocompatibility, non-toxic profile, and stability towards variation in temperature (Gaaz et al., 2015). In this project, our goal was to develop a SARM-loaded PLGA microparticle formulation capable of providing in vitro and in vivo release of SARM over at least 4 weeks. An acid terminated PLGA polymer was selected with 50:50 (L:G) monomer ratio to encapsulate SARM and decided to produce SARM-loaded PLGA microparticles with two different particle sizes, that have different formulation characteristics (particle size, drug loading, encapsulation efficiency, and in vitro release profiles), to be subsequently tested for its in vivo release profile and pharmacokinetics in mice; and finally test its ability to reverse hyperalgesia in a wide-spread pain (fibromyalgia) animal model (Sluka et al., 2001). To produce SARM-loaded PLGA microparticles with two different particle sizes, all the formulation parameters were fixed while the emulsification speed and time were varied using two different emulsification instruments. Formulation 1 was made with an overhead paddle stirrer which spun at 2883 rpm for 4 minutes, while formulation 2 was produced with a homogenizer set at 9500 rpm for 1 minute.

### Characterization of SARM-Loaded PLGA Microparticles

### Particle Size and Surface Morphology

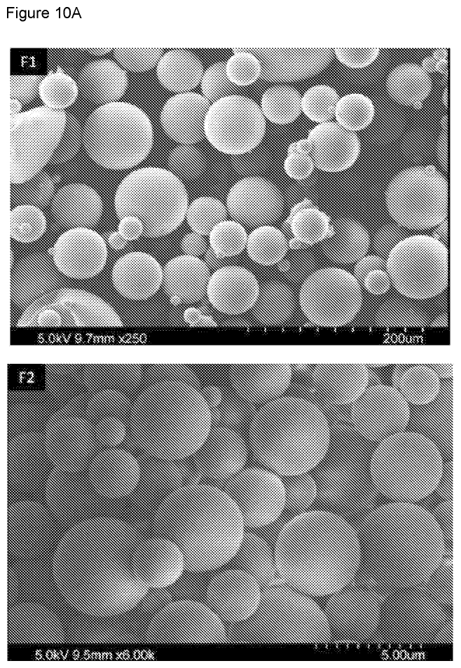

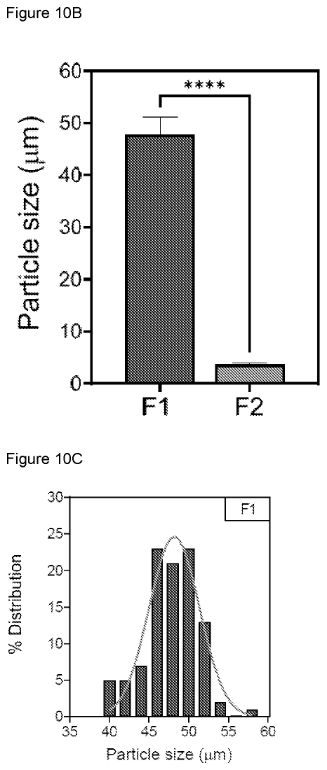

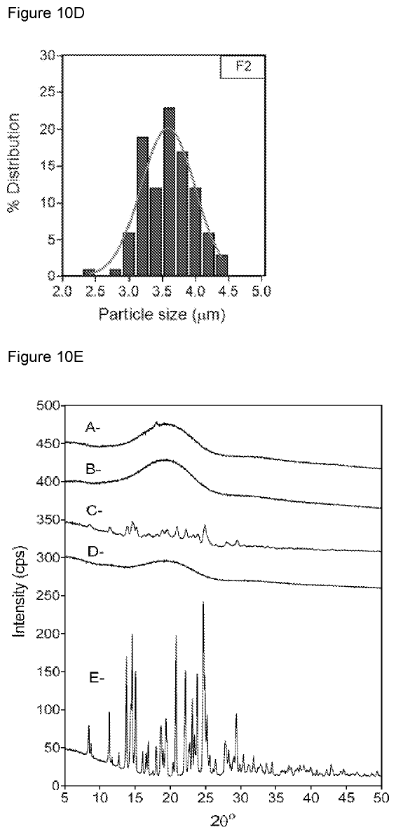

Scanning electron microscopy photomicrographs (FIG. 10A) show the particles to have been spherical with a smooth, non-porous surface and no observable un-encapsulated drug crystals. SARM-loaded PLGA microparticles (F1 and F2) had significantly different particle sizes (p<0.01, n=100). The average diameters (±SD) of F1 and F2 were 47.7 μm±3.4, and 3.6 μm±0.38, respectively (FIG. 10B). This significant difference in particle size between F1 and F2 was due to using a higher emulsification speed in F2 (9500 rpm, using a homogenizer) as compared to a lower emulsification speed in F1 (2833, using an overhead stirrer). The higher emulsification speed for longer time breaks down the inner oil phase into smaller droplets, due to the higher shear force input into the system and the long duration of time, resulting in a smaller particle size after evaporation of the organic solvent (Yang & Owusu-Ababio, 2000). Particle size frequency distribution histograms (FIGS. 10C-10D) showed a homogenous, unimodal particle size distribution for both formulations. Span values for both formulations were low (0.09 for F1 and 0.15 for F2), indicating a narrow spread of particle sizes within each formulation.

Differential Scanning calorimetry (DSC)

DSC helps investigate whether there is an interaction between the polymer and the encapsulated drug by evaluating any change or disappearance of the glass transition temperature (Tg) of the polymer or the melting point (m.p.) of the encapsulated drug (Bragagni et al., 2018; Chen et al., 2004). FIG. 7 shows DSC thermograms of SARM, PLGA (50:50), physical mixture of SARM and PLGA, SARM microparticles (F1 and F2). The thermogram of pure SARM demonstrated a sharp endothermic peak at 134.18° C. (range 127.74-138.61° C.) corresponding to the m.p. of its crystalline form. PLGA 50:50 showed a brief endothermic band at 49.79° C. corresponding to its Tg (Lesnak et al., 2020). The physical mixture of pure SARM and the PLGA polymer showed both, the brief endothermic peak of PLGA at 48.91° C., corresponding to its Tg and a sharp endothermic peak at 134.18° C. corresponding to the m.p of SARM, although the SARM endothermic peak was weaker compared to the pure drug which could be due to the smaller amount of SARM in the physical mixture and its dilution by the polymer. Finally, the SARM microparticles (F1 and F2) showed complete disappearance of the SARM endothermic peak, which can indicate that there is an interaction between the polymer and SARM in the microparticle formulation, and that the SARM is likely present in an amorphous, molecularly dispersed form inside the microparticles.

### Powder X-Ray Diffraction (PXRD)

Powder X-ray diffraction (PXRD) is a useful tool to determine whether material is in a crystalline or amorphous state. When a crystalline drug is encapsulated into a microparticle formulation it loses its crystalline properties and exists in a molecularly dispersed (amorphous) form (Vysloužil et al., 2016). This makes PXRD a useful tool to confirm the encapsulation of the drug into the microparticle formulation. FIG. 10E shows the x-ray diffraction patterns of SARM, PLGA polymer (503H), a physical mixture of SARM and PLGA polymer, and SARM-loaded microparticles (F1 and F2). The diffractogram of pure SARM (FIG. 10E) showed sharp, intense peaks characteristic of SARM in the crystalline state. On the other hand, due to the amorphous nature of the PLGA polymer, its diffractogram (FIG. 10E) showed a broad band with no sharp peaks confirming its existence in an amorphous state. The physical mixture diffractogram was simply a superposition of SARM and PLGA polymer diffractogram, indicating no interaction between the polymer and SARM and that the drug is still in its crystalline state. Finally, the diffraction patterns of the SARM-loaded PLGA microparticles (F1 and F2) (FIG. 10E) showed complete disappearance of the sharp peaks (characteristic to SARM crystalline state) which indicates that SARM has lost its crystalline state due to encapsulation into the PLGA microparticle formulation and is present in an amorphous molecularly dispersed form.

### In Vitro Release Kinetics

PLGA is a bulk (homogenous) eroding biopolymer where drug release from the microparticles is driven by a combined diffusion-erosion mechanism that typically follows a biphasic or triphasic pattern. At first, an initial rapid burst release over the first 4 days is observed due to the diffusion/dissolution of the drug near or at surface of the microspheres. This is followed by a slow-release lag-phase over day 4 to day 18 which occurs due to the time needed for build-up of acidic degradation moieties, resulting from the hydrolytic cleavage of the PLGA ester bonds, to reach sufficient concentrations before the bulk-erosion phase of the polymer is initiated (Han et al., 2016). The in vitro release of both SARM-loaded microparticle formulations showed a typical bi-phasic release pattern characteristic of PLGA (FIG. 10F) although in F1 the release pattern is likely following a tri-phasic release with an initial fast burst release followed by a slow phase then a fast release phase. The in vitro cumulative release profile of SARM from F2 was faster than that of F1. After 3 days, 32.3% (±3.5) of SARM was released from F2 vs 19.9% (±5.6) from F1. This was followed by a slow-release phase between day 3 and 18 and a faster release phase between day 18 and day 25. By day 25, F2 has released 75.5% (±7.05) of the encapsulated SARM vs 58% (±12.6) released from F1. The overall faster release rate of SARM from F2 is likely due to the significantly smaller average particle size of F2 versus F1 (3.6 μm versus 47.7 μm). For particles with a smaller average diameter, the encapsulated drug needs to travel a shorter distance before being released from the particles, in addition, the total surface area of particles per unit volume of release medium is much higher in particles with a smaller average diameter (Khaled et al., 2010). Based on the in vitro release kinetics data, we selected formulation 1 to further evaluate its in vivo release properties and pharmacokinetics in mice and its ability to reverse hyperalgesia in a fibromyalgia (widespread pain) animal model. F1 was chosen over F2 because of the overall slower in vitro release profile of F1; indicating that it was more likely to meet a target 4-week in vivo release of SARM. In vivo release of encapsulated drugs from microparticles is likely to be faster due to the action of enzymes in the body that can result in faster degradation of PLGA (Lu et al., 2011).

### In Vivo Release and Pharmacokinetics

FIGS. 4 (A and B) shows the plasma concentration vs time profile of SARM following the administration of SARM MP F1 and F2 to male and female mice (n=16, n=8/formulation, n=4/male or female) at Day 0 and Day 8 of the study at a dose of 200 mg/kg SARM as described in the methods section. FIG. 4A shows the pooled data from male and female mice and FIG. 4B shows data stratified by sex where no significant difference was observed between male and female mice. F1 has shown a slower, more sustained release than that of F2. Due to the smaller particle size diameter of SARM F2 microspheres than that of F1, SARM was released faster from F2 that from F1. SARM plasma levels have peaked rapidly at day 8 and started to decline after that timepoint following the administration of F2 and SARM was not detected in plasma after day 22 in this group. While, F1 has shown a more sustained release and SARM plasma levels were detectable up to day 29. This data indicates that the larger particle size microspheres of F1 could provide sustained plasma levels of SARM over a period of 1 month and it was selected to study its effect in reversing hyperalgesia in a widespread pain mouse model. In addition, SARM observed plasma levels from F1 were between 2.354-55.7 μg/mL which is within a therapeutically relevant concentration when compared to SARM plasma levels observed after IV or PO administration to rats as reported in literature (Di Wu et al, 2006).

### SARM-Loaded Microparticles Alleviates Muscle Hyperalgesia in 2 Injections

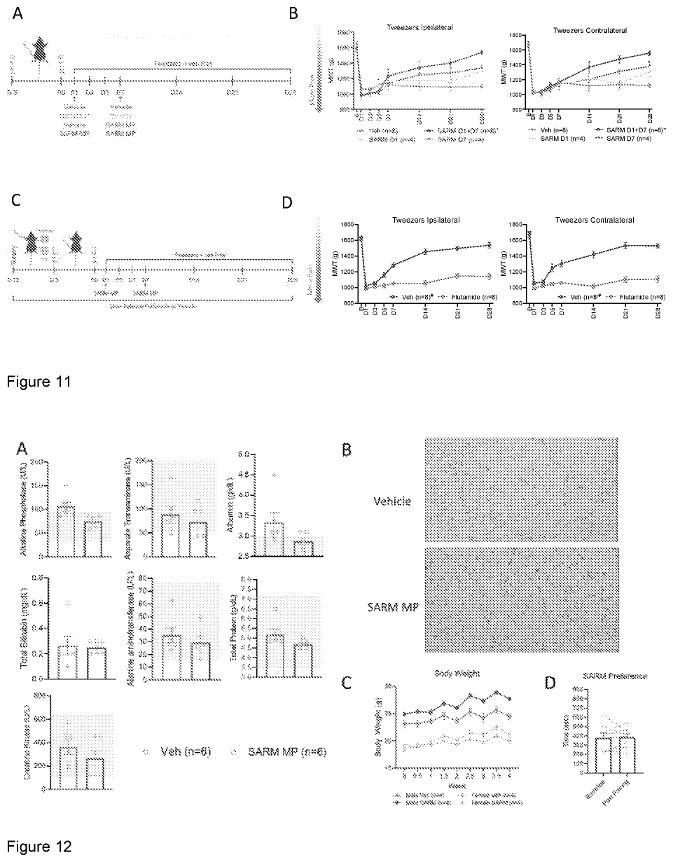

To test if SARM-loaded PLGA microparticles (F1) could alleviate muscle hyperalgesia the acidic saline muscle pain model was used and the presence of muscle hyperalgesia and paw allodynia on day 1 was confirmed after model induction. Animals then received subcutaneous injections of SARM-loaded PLGA microparticles (60 mg) or its vehicle on days 1 and 7 after induction of the pain model. Animals were randomly grouped to receive two injections of SARM loaded PLGA microparticles on days 1 and 7 (n=4 male, n=4 female), a single injection of SARM loaded PLGA microparticles (Day 1: n=2 male, n=2 female; Day 7: n=2 male, n=2 female), or two injections of SARM microparticle vehicle on day 1 and 7 (n=4 male, n=4 female). Muscle hyperalgesia and paw allodynia was reassessed on days 3, 5, 7, 14, 21, and 28 after pain model induction (FIG. 11A). There was a significant time by group interaction on both the ipsilateral (F_{18.114}=4.22, p<0.01) and contralateral (F_{18.114}=5.36, p<0.01) limb for MWT values. Post hoc testing demonstrated that animals who received two microparticle injections had a reversal of muscle hyperalgesia on the ipsilateral (p<0.01) (FIG. 11B) and contralateral (p<0.01) (FIG. 12B) limb when compared with animals who received two vehicle injections. Post hoc testing also revealed no significant differences in MWT values between animals who received a single SARM microparticle injection and two vehicle injections on both the ipsilateral (p=0.12-0.85) and contralateral (p=−0.13-0.86) limb. The SARM microparticles did not produce a time by group interaction for paw allodynia on the ipsilateral (F_{18.114}=0.99, p=0.47) or contralateral paw (F_{18.114}=1.05, p=0.41). This demonstrates that a SARM-loaded PLGA microparticle formulation can alleviate muscle hyperalgesia following 2 injections.

### SARM-Loaded Microparticles Stimulate Analgesia Through Activation of Androgen Receptors

To determine if SARM are producing analgesic effects through activation of androgen receptors slow release flutamide (n=4 male, n=4 female) or vehicle (n=4 male, n=4 female) pellets were subcutaneously implanted. The acidic saline muscle pain model was induced and the presence of muscle hyperalgesia and paw allodynia 1 day after model induction was confirmed. All animals then received SARM-loaded PLGA microparticles (60 mg) subcutaneously on days 1 and 7 after pain model induction. Muscle hyperalgesia and paw allodynia was reassessed on days 3, 5, 7, 14, 21, and 28 after pain model induction (FIG. 11C). Animals with flutamide pellets did not demonstrate an alleviation of muscle hyperalgesia, measured via MWT, when compared with animals who received control pellets on both the ipsilateral (F_{6.78}=18.13, p<0.01) and contralateral limb (F_{6.78}=12.64, p<0.01) (FIG. 3D). Again, SARM was unable to alleviate paw allodynia and therefore there were no significant differences in von Frey response values between animals who received flutamide or control pellets on both the ipsilateral (F_{6.78}=0.26, p=0.95) and contralateral (F_{6.78}=1.07, p=0.38) paw. This data demonstrates that SARM are working through activation of androgen receptors to alleviate muscle hyperalgesia.

### SARM Toxicity and Rewarding Behavior

To test for toxicity, following the completion of 4-week behavior testing serum was collected from the animals receiving two injections of SARM-loaded microparticles (n=3 male, n=3 female) or its vehicle (n=3 male, n=3 female) and liver and cardiac toxicity panels analyzed. No significant difference was found in levels of alkaline phosphatase, aspartate transaminase, alanine aminotransferase, total bilirubin, albumin, total protein, and creatine kinase when vehicle-treated and microparticle-treated groups were compared (p=0.02-0.82) (FIG. 12A). Examination of the heart tissues following four weeks during of exposure did not show differences between mock and treatment groups (FIG. 12B). Body weight was measured throughout the duration of the 4-week in vivo hyperalgesia treatment experiment. No significant differences were found in body weights between animals receiving two injections of the SARM-loaded microparticles when compared with those receiving two vehicle injections (F_{1.14}=1.19, p=0.29) (FIG. 12C). Lastly, to determine if SARM produced reward like behavior, conditioned place preference paradigm testing was utilized. Animals were paired with soluble SARM (25 mg/kg) (n=4 male, n=4 female) daily for 5 days. Animals did not show an increased preference for the SARM paired chamber between baseline (380.3±51.0 sec) and post drug pairing testing (385.7±37.1 sec) (t₇=0.15, p=0.88) (FIG. 12D). Together, this data indicates that the SARM-loaded PLGA microparticles did not induce any cardiac or liver toxicity and that soluble SARM did not produce reward like behavior in mice.

### Discussion

The current study is the first to show that systemic administration of a SARM can be used to alleviate widespread muscle hyperalgesia using an animal model. A PLGA microparticle formulation loaded with the SARM was prepared that produced long-term release, in vitro and in vivo, and long-term reduction in hyperalgesia. The reduction in hyperalgesia by the SARM microparticle formulation was prevented by flutamide, suggesting that the analgesic effect of SARM works through activation of androgen receptors. Repeated SARM administration did not produce rewarding behavior using a conditioned place preference paradigm. There were no changes in body weight, cardiac and liver toxicity panels, heart histology in animals treated with SARM-microparticles suggesting that the formulation was safe. Thus, the disclosure provides for a slow-release microparticle formulation of a SARM that reduces hyperalgesia and is non-addictive and safe.

While this may be the first use SARM for muscle pain relief, this work is in agreement with animal and clinical studies showing analgesic effects of testosterone. In animals, testosterone reduces hyperalgesia in models of muscle, temporomandibular, formalin-induced, and stress-induced pain (Lesnak et al., 2020; Fanton et al., 2017; Ji et al., 2018; Gaumond et al., 2005). Clinically in women with fibromyalgia (n=12) treated with testosterone for 4 weeks showed a reduction in muscle pain, stiffness, and fatigue when compared to pre-treatment ratings (White et al., 2015). There are mixed results in hypogonadal or androgen deficient men with some showing testosterone treatment reduces pain (Basaria et al., 2015; Kato et al., 2020), while others showing no effect (Glintborg et al., 2020). While SARM administration reversed muscle hyperalgesia, it was unable to alleviate the paw hyperalgesia. As the SARM used in the current study targets muscle and bone androgen receptors (Mohler et al., 2009; Chen et al., 2005; Gao et al., 2006), it is possible that it is more effective for muscle pain than cutaneous pain. Alternatively, the SARM could target the site of insult over secondary sites; however, a reduction in the contralateral, uninjured muscle was observed, suggesting the SARM works to reduce hyperalgesia outside the site of insult.

SARM produced analgesia through activation of androgen receptors. Androgen receptors are located throughout the body, including both peripheral and central nociceptive sites, and on immune cells that modulate nociception (Davey & Grossmann, 2016). Since the SARM was administered systemically, it is unclear what the site of action of the SARM is. Activation of androgen receptors transcriptionally increases expression of mu-opioid and cannabinoid type-1 receptors on nociceptors, both of which have endogenous anti-nociceptive effects (Lee et al., 2013; Lee et al., 2016; Zhang et al., 2014; Nui et al., 2012). Activation of androgen receptors also decreases pro-inflammatory cytokines (Bianchi, 2019; Malkin et al., 2004; D'Agostino et al., 1999; Bebo et al., 1999), increases anti-inflammatory cytokines (Malkin et al., 2004; D'Agostino et al., 1999; Bebo et al., 1999; Mohamad et al., 2019; Liva et al., 2001) from a variety of immune cells, and can polarize resident macrophages to an M2 anti-inflammatory phenotype (Becerra-Diaz et al., 2020; Becerra-Diaz et al., 2018). Prior studies show anti-inflammatory cytokines like IL-10 and IL-4, are analgesic (Leung et al., 2016; Kiguchi et al., 2015). Lastly, lower amounts of circulating testosterone are associated with lower activation the rostral ventromedial medulla, a brain region responsible for descending pain inhibition (Vincent et al., 2013). Since, androgen receptors are located in a diverse range of tissues, it likely the analgesic effects of SARM are multifactorial.

SARMs have a half-life between 16-24 hours which suggests that daily administration is necessary to maintain therapeutic plasma concentrations that will reverse muscle pain (Naafs, 2018). Daily administration results in lower adherence when compared to weekly or monthly administration (Jimmy & Jose, 2011; Sampaio et al., 2020; Brown & Bussell, 2011) and could result in failure of SARM to produce its analgesic effects. For those with chronic diseases, long-term release of pharmaceutical agents can improve adherence and improve symptom and disease management (Romley et al., 2020). Several studies have reported non-adherence to medication in patients with chronic pain and have indicated various reasons for non-adherence, including side effects, dosing frequency, polymedication, and both high and low pain intensity (Sampaio et al., 2020; Timmerman et al., 2016; Kipping et al., 2014). Injectable polymeric long-acting formulations are clinically available, safe, and have the potential to overcome adherence issues that result from repeated administration of medications (Park et al., 2021; Park et al., 2019; Romley et al., 2020; Yun et al., 2015; Ahmed et al., 2016). Thus, an injectable long-acting SARM-loaded PLGA microparticle containing formulation can be injected subcutaneously to provide sustained release and maintain effective plasma levels of SARM over a long period of time, and thus eliminate the need for daily administrations.

The current study determined that a larger microparticle produces a long-term release in plasma of mice. This size particle is similar to currently available compounds that are delivered subcutaneously through 20-21-gauge needles and include Trelstar® (21-G), Risperidal Consta® (21-G) and Vivtrol® (20-G) (Park et al., 2019). The long-term release, at least 35 days, with accompany analgesia suggests adequate plasma levels can be achieved with a single or twice per month administration. Thus, the microparticle formulation has the potential to produce long-term reduction in pain.

The lack of toxicity in the current study for SARMs agrees with prior work in both animals and humans. The weight of the prostate and seminal vesicle was unchanged after administration of SARMs to orchiectomized rats when compared to gonadally intact male rats suggesting a lack of activation of androgenic signaling (Gao et al., 2005). Similarly, no adverse androgenic events such as virilization, hirsutism, and negative effects on prostate have been reported in randomized double-blind placebo controlled clinical trials in both male and female human subjects (Dalton et al., 2011). Numerous clinical studies examined adverse events and report the following: constipation, dyspepsia, nausea, muscle soreness, headaches, fatigue, increased hemoglobin and creatine kinase, and decreased high density lipoproteins (HDL) (Dalton et al., 2011; Clark et al., 2017; Bhattacharya et al., 2016). The most consistently cited adverse event with SARM treatment is an increase in the liver enzyme alanine-aminotransferase (ALT) (Dalton et al., 2011; Clark et al., 2017; Bhattacharya et al., 2016; Dobs et al., 2013). However, we found no alterations in ALT in the current study in response to SARM-loaded microparticle treatment. Differences could be related to the microparticle formulation's ability to maintain stable levels, a shorter duration of treatment, or species differences between the mice and human studies. There are also published case reports of liver injury due to SARM misuse in young, healthy individuals (Barbara et al., 2020; Flores et al., 2020; Koller et al., 2021). Since SARM have anabolic effects that increase muscle growth and enhance athletic performance, they have become a drug of interest for athletes and weightlifters. In these case reports, the dose of SARM utilized was either unreported or was 10 to 100 times higher than that utilized in previous clinical trials (Barbara et al., 2020; Flores et al., 2020; Killer et al., 2021). Thus, SARM should still be viewed as safe when taken at appropriate doses.

This work demonstrates activation of androgen receptors through SARM as a new treatment of chronic muscle pain. Activation of androgen receptors utilizing testosterone are analgesic in both pre-clinical and clinical research (Lesnak, et al., 2020; Fanton et al., 2017; White et al., 2015). However, SARM appear to be better tolerated with fewer adverse side effects due to minimal activation of androgenic signaling (Dalton et al., 2011; Gao et al., 2005). SARM-loaded PLGA microparticles produced long-lasting release and microparticle-based drug delivery could be ideal for individuals with chronic pain and could improve patient convenience, increase adherence, and optimize maintenance of therapeutic concentrations for a variety of therapeutics.

The SARM utilized was designed to target skeletal muscle and bone tissue. Several SARMs have been developed that serve as tissue specific androgen receptor agonists at other tissue sites such as nervous system tissue. SARMs may be analgesic in other pain models such as neuropathic or inflammatory type pain. The use of the tested formulation achieved full reversal of hyperalgesia with two doses. Other doses and/or other particle sizes may produce a similar effect, e.g., with a single administration.

### REFERENCES

Ahmed et al., J. Pharm. Sci., 105:1173 (2016).
Barbara et al., ACG Case Rep. J., 7:e00370 (2020).
Basaria et al., Pain, 156:280 (2015).
Bebo, Jr., et al., J. Immunol., 162:35 (1999).
Becerra-Diaz et al., Front. Immunol., 11:1698 (2020).
Becerra-Diaz et al., J. Immunol., 201:2923 (2018).
Bhattacharya et al., Clin. Thera., 38:1401 (2016).
Bianchi, J. Endocr. Soc., 3:91 (2019).
Bragagni et al., Int. J. Pharm., 547:24 (2018).
Brown & Bussell, Mayo Clin. Proc., 86:304 (2011).
Chen et al., Endocrinology, 146:5444 (2005).
Chen et al., J. Biomed. Mater. Res. A, 70:412 (2004).
Clark et al., Br. J. Clin. Pharma., 83:2179 (2017).
Crawford et al., Curr. Oncol. Rep., 18:37 (2016).
Crawford et al., Curr. Oncol. Rep., 18:37 (2016).
D'Agostino et al., Ann. N. Y. Acad. Sci., 876:426 (1999).
Dalton et al., J. Cachexia Sarcopenia Muscle, 2:153 (2011).
Dalton et al., J. Cachexia Sarcopenia Muscle, 2:153 (2011).
Davey & Grossmann, Clin. Biochem. Rev., 37:3 (2016).
Dobs et al., Oncology, 14:335 (2013).
Fanton et al., Pharmac. Biochem. Beh., 152:30 (2017).
Flores et al., Hepatol. Commun., 4:450 (2020).
Gaaz et al., Molecules, 20:22833 (2015).
Gao et al., Endocrinology, 145:5420 (2004).
Gao et al., Endocrinology, 146:4887 (2005).
Gao et al., Endocrinology, 146:4887 (2005).
Gao et al., Pharm. Res., 23:1641 (2006).
Gaskin & Richard, J. Pain, 13:715 (2012).
Gaumond et al., Brain Res., 1052: 105 (2005).
Glintborg et al., Eur. J. Endocrinol., 182:539 (2020).
Han et al., Front Pharmacol., 7:1 (2016).
Han et al., Frontiers in Pharmacology, 7:1 (2016).
Handlon et al., ACS Med. Chem. Lett., 7:83 (2016).
Iglay et al., Clin. Ther., 37:1813 (2015).
Ji et al., J. Pain, 19:764 (2018).
Jimmy & Jose, Oman Med. J., 26:155 (2011).
Johnston et al., Adv. Ther., 31:1119 (2014).
Kato et al., Andrologia, 52:e13768 (2020).
Kearbey et al., Pharmaceutical Res., 24:328 (2007).
Khaled et al., AAPS PharmSciTech, 11:859 (2010).
Kiguchi et al., Pain, 156:684 (2015).
Kipping et al., Pain Physician, 17:81 (2014).
Koller et al., World J. Clin. Cases, 9:4062 (2021).
Koyanagi et al., Front. Pharmacol., 7:212 (2016).
Lagreca et al., Prog. Biomater., (2020).
Lee et al., Neuroscience, 254:395 (2013).
Lee et al., Neuroscience, 331:52 (2016).
Lesnak et al., Pain, _:_ (2020).
Lesnak et al., Pain, _:_ (2022).
Leung et al., Pain, 157:70 (2016).
Liva & Voskuhl, J. Immunol., 167:2060 (2001).
Lu et al., Int. J. Pharm., 418: 142 (2011).
Malkin et al., J. Clin. Endocrinol. Metab., 89:3313 (2004).
Malmenäs et al., Clin. Thera., 35:795 (2013).
Medic et al., Neuropsychiatr. Dis. Treat., 9:119 (2013).
Miklos et al., Acta Marisiensis-Seria Medica, 65:49 (2019).
Mohamad et al., Aging Male, 22:129 (2019).
Mohler et al., J. Med. Chem., 52:3597 (2009).
Mohler et al., J. Med. Chem., 52:3597 (2009).
Naafs, Open Access J. Reprod. Sys. Sex. Dis., 1:1 (2018).
Naguib et al., Drug Deliv. Transl. Res., _:_ (2020).
Naguib et al., Drug Deliv. Transl. Res., 11:182 (2021).
Naguib et al., Mol. Pharm., 17:3643 (2020).
NCBI, pp. PubChem Compound Summary for CID 11326715, Enobosarm. (2022).
Niu et al., Pain, 153:2283 (2012).
Osterberg & Blaschke, New Eng. J. Med., 353:487 (2005).
Park et al., J. Control Release, _:_ (2020).
Park et al., J. Control Release, 304:125 (2019). Available from:
https://doi.org/10.1016/j.jconrel.2019.05.003
Park et al., J. Control. Release, 304:125 (2019).
Park et al., J. Control. Release, 329: 1150 (2021).
Petering & Brooks, Am. Fam. Physician, 96:441 (2017).
Romley et al., J. Gen. Intern. Med., 35:354 (2020).
Sampaio et al., Patient Prefer Adher., 14:321 (2020).
Skyba et al., J. Pain, 6:41 (2005).
Sluka & Rasmussen, Pain, 148:188 (2010).
Sluka et al., Muscle & Nerve, 24:37 (2001).
Sluka et al., Muscle & Nerve, 24:37 (2001).
Solomon et al., Sex. Med. Rev., 7:84 (2019).
Solomon et al., Sex. Med. Rev., 7:84 (2019).
Su et al., Drug Deliv., 28:1397 (2021).
Timmerman et al., Acta Anaesth. Scand., 60:416 (2016).
Tunceli et al., Patient Prefer. Adherence, 9:191 (2015).
Vincent et al., Pain, 154:515 (2013).
Vysloužil et al., Pharm. Dev. Technol., 21:214 (2016).
Wafa et al., J. Pharma. Exp. Thera., 370:855 (2019).
White et al., Int. Immunopharmacol., 27:249 (2015).
White et al., Int. Immunopharmacol., 27:249 (2015).
Wu et al., Drug Metab. Dispos., 34:483 (2006).
Yang & Owusu-Ababio, Drug Dev. Ind. Pharm., 26:61 (2000).
Yokoyama et al., J. Pain, 8:422 (2007).
Yun et al., J. Control. Release, 219:2 (2015).
Zhang et al., Eur. J. Pain (London, England), 18:151 (2014).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A sustained release composition comprising one or more particles comprising a polymer and an amount of a select androgen receptor modulator (SARM) and a pharmaceutically acceptable carrier;
wherein:
the particles comprise a synthetic polymer, the synthetic polymer comprising 85 to 95% poly lactic-co-glycolic acid (PLGA).

2. (canceled)

3. (canceled)

4. (canceled)

5. (canceled)

6. (canceled)

7. The composition of claim 1, wherein the particles are microparticles.

8. The composition of claim 7 wherein the microparticles have a diameter of about 1 to about 100 microns.

9. (canceled)

10. (canceled)

11. (canceled)

12. (canceled)

13. The composition of claim 1, wherein the SARM comprises C-6, S-23, BA321, FL442, MK-45412, LGD226, S-40542, S-1, S-4,
GLPG0492, GTx-024 (enobosarm), LY2452473, GSK2881078, GSK2849466, PF-06260414, or LGD-4044.

14. (canceled)

15. The composition of claim 1, wherein the SARM is released for up to 10 weeks.

16. (canceled)

17. (canceled)

18. (canceled)

19. The composition of claim 1, which is injectable.

20. A method to prevent, inhibit or treat pain in a mammal, comprising: administering to a mammal in need thereof an effective amount of the composition of claim 1.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. The method of claim 20, wherein the composition is administered weekly.

34. (canceled)

35. (canceled)

36. A method to prevent or treat low testosterone levels in a mammal, comprising: administering to a mammal in need thereof an effective amount of the composition of claim 1.

37. (canceled)

38. (canceled)

39. (canceled)

40. The method of claim 36, wherein the composition is injected.

41. (canceled)

42. (canceled)

43. (canceled)

44. The method of claim 36, wherein the composition is administered weekly.

45. (canceled)

46. The composition or method of claim 1, wherein the SARM comprises a compound having a non-steroidal structure.

47. The composition or method of claim 1, wherein the SARM comprises a compound according to Formula I, or a pharmaceutically acceptable salt thereof: 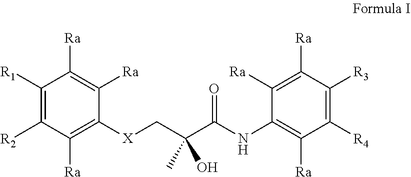
wherein
R₁ is F, Cl, Br, I, CF₃, CN, NO₂, NHCORₐ; CONRₐRₐ, SORₐ, or SO₂Rₐ;
R₂ is H, F, Cl, Br, I, or CF₃;
Rₐ is F, Cl, Br, I, NO₂, CN, CF₃, CONRₐRₐ, SORₐ, or SO₂Rₐ;
R₄ is H, F, Cl, Br, I, or CF₃;
Ra is, in each instance, independently H, F, Cl, Br, I, CF₃, optionally substituted C₁-C₄ alkyl, or optionally substituted C₁-C₄ heteroalkyl; and
X is O, S, NH, SO₂, SO, CH₂, or CO.

48. (canceled)

49. (canceled)

50. (canceled)

51. (canceled)

52. (canceled)

53. (canceled)

54. (canceled)

55. The composition or method of claim 47, wherein the compound Formula I has the structure: 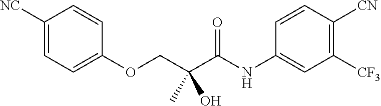 [SMILES: C[C@](O)(COc1ccc(C#N)cc1)C(=O)Nc1ccc(C#N)c(C(F)(F)F)c1]
or a pharmaceutically acceptable salt thereof.

56. (canceled)

57. The composition of claim 1, wherein the SARM comprises a compound according to Formula II, or a pharmaceutically acceptable salt thereof: 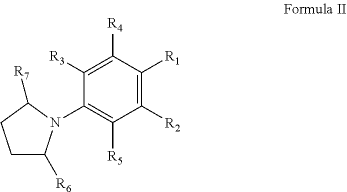
wherein
R¹ is F, Cl, Br, I, SRₐ, NO₂, CN, CF₃, CONRₐRₐ, SORₐ, or SO₂Rₐ;
R² is F, Cl, Br, I, SRₐ, NO₂, CF₃, CORₐ, CO₂Rₐ, CONRₐRₐ, SORₐ, SOzRa, SO₂NRₐRₐ, NHCORₐ, NHCONRₐRₐ, or optionally substituted C₁-C₄ alkyl,
R³ and R⁴ are each independently H, F, Cl, Br, I, ORₐ, CF₃, or optionally substituted C₁-C₄ alkyl;
R⁵ is H, F, Cl, Br, I, CF₃, or optionally substituted C₁-C₄ alkyl;
R⁶ is optionally substituted C₁-C₈ alkyl, optionally substituted C₂-C₈ alkenyl, CF₃, optionally substituted C₂-C₈ haloalkenyl, optionally substituted C₂-C₈ heteroalkenyl, optionally substituted C₂-C₈ alkynyl, optionally substituted C₂-C₈ haloalkynyl, optionally substituted C₂-C₈ heteroalkynyl, optionally substituted heteroaryl, optionally substituted aryl, optionally substituted (CH₂)-heteroaryl, optionally substituted (CH₂)-aryl, CH(Rₐ)ORₐ, CH(Rₐ)NRₐRₐ, CORₐ, or Y;
R7 is H, F, CF₃, optionally substituted C₁-C₆ alkyl, optionally substituted C₁-C₆ heteroalkyl, optionally substituted C₂-C₆ alkynyl, and optionally substituted C₂-C₆ alkenyl;
Rₐ is, in each instance, independently H, F, CF₃, optionally substituted C₁-C₄ alkyl, or optionally substituted C₁-C₄ heteroalkyl; and
Y is —CH(OH)CF₃ having an (R)-stereocenter, and which provides an (R)-stereocenter at the carbon to which it is attached.

58. (canceled)

59. (canceled)

60. (canceled)

61. (canceled)

62. (canceled)

63. The composition or method of claim 57, wherein the compound Formula II has the structure: 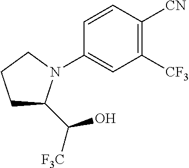 [SMILES: N#Cc1ccc(N2CCC[C@@H]2[C@@H](O)C(F)(F)F)cc1C(F)(F)F]
or a pharmaceutically acceptable salt thereof.

64. (canceled)

65. The composition of claim 1, wherein the SARM comprises a compound according to Formula III, or a pharmaceutically acceptable salt thereof: 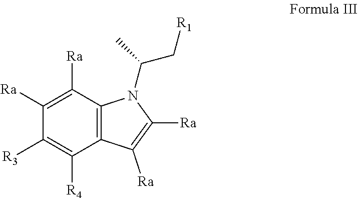
wherein
Ra is, in each instance, independently H, H, F, Cl, Br, I, CF₃, optionally substituted C₁-C₄ alkyl, or optionally substituted C₁-C₄ heteroalkyl;
R¹ is NHCOR_{b}, COR_{b}, CONRₐR_{b}, SOR_{b}, or SO₂R_{b};
R³ is CN or NO₂;
R⁴ is H, F, Cl, Br, I, or CF₃; and
Rb is F, CF₃, CN, or optionally substituted C₁-C₄ alkyl.

66. (canceled)

67. The composition or method of claim 65, wherein the compound Formula III has: 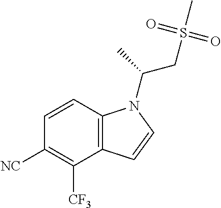 [SMILES: C[C@H](CS(C)(=O)=O)n1ccc2c(C(F)(F)F)c(C#N)ccc21]
or a pharmaceutically acceptable salt thereof.

68. (canceled)

69. The composition claim 1, wherein the SARM comprises a compound according to Formula IV, or a pharmaceutically acceptable salt thereof: 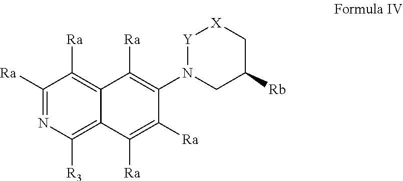
wherein
Ra is H, F, Cl, Br, I, CF₃, optionally substituted C₁-C₄ alkyl, or optionally substituted C₁-C₄ heteroalkyl;
R³ is F, Cl, Br, I, NO₂, CN, CF₃, CONRₐRₐ, SORₐ, or SO₂Rₐ;
Rb is F, Cl, Br, I, CF₃, optionally substituted C₁-C₄ alkyl, or optionally substituted C₁-C₄ heteroalkyl;
X is CH2, absent, or NH; or
Y is NHCOR_{b}, COR_{b}, CONRₐR_{b}, SOR_{b}, or SO₂R_{b}.

70. (canceled)

71. The composition or method of claim 69, wherein the compound Formula IV has the structure: 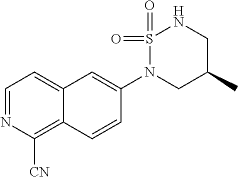 [SMILES: C[C@@H]1CNS(=O)(=O)N(c2ccc3c(C#N)nccc3c2)C1]
or a pharmaceutically acceptable salt thereof.

72. (canceled)
